(19) United States
(12) Patent Application Publication
(10) Pub. No.: US 2025/0230451 A1
(43) Pub. Date: Jul. 17, 2025

(54) COMPOSITION COMPRISING GENE EDITING PROTEIN FOR CANNABIS SATIVA GENE EDITING, AND GENE EDITING METHOD USING SAME

(71) Applicant: GENKORE INC., Daejeon (KR)

(72) Inventors: Yong-Sam KIM, Daejeon (KR); Suk Weon KIM, Daejeon (KR); Do Yon KIM, Daejeon (KR); Seyeon PARK, Daejeon (KR); Yuhee CHUNG, Daejeon (KR)

(21) Appl. No.: 18/853,825

(22) PCT Filed: Apr. 5, 2023
(86) PCT No.: PCT/KR2023/004622

§ 371 Date: Oct. 3, 2024

(30) Foreign Application Priority Data
Apr. 5, 2022 (KR) 10-2022-0042377

(51) Int. Cl. C12N 15/82; A01H 4/00; C12N 9/02; C12N 9/22; C12N 15/11

(52) U.S. Cl. CPC C12N 15/8201; A01H 4/005; C12N 9/001; C12N 9/22; C12N 15/11; C12N 2310/20; C12Y 103/9903; C12Y 121/03007; C12Y 121/03008; C12Y 121/99

(57) ABSTRACT

Provided are a composition for editing a Cannabis gene, a kit for editing a Cannabis gene, a kit for inhibiting Cannabis DNA expression and targeting the DNA, and a method of editing Cannabis DNA, each using a gene editing protein; a method of preparing an edited Cannabis gene including a step of forming calli; a gRNA for editing a Cannabis gene; and a use of the gRNA of the present invention and a use of both the gene editing protein and the gRNA for editing a Cannabis gene.

### TECHNICAL FIELD

The present invention relates to a composition for editing a Cannabis gene, a kit for editing a Cannabis gene, a kit for inhibiting Cannabis DNA expression and targeting the DNA, and a method of editing Cannabis DNA, each using a gene editing protein; a method of preparing a gene-edited Cannabis including a step of forming calli; a gRNA for editing a Cannabis gene; and a use of the gRNA of the present invention and a use of both the gene editing protein and the gRNA for editing a Cannabis gene.

### BACKGROUND ART

Genetic scissors are a biotechnology technique enabling knocking-out of a function of a target gene by cleaving a specific site of the gene or knocking-in, which is a genetic technique for replacing a specific gene sequence with a desired sequence. The CRISPR-Cas system, as a third-generation genetic scissors developed through first-generation ZFN and second-generation TALEN, is widely used because there is no need to create a DNA binding region every time and specificity is excellent (Zhang, F., et al., CRISPR/Cas9 for genome editing: progress, implications, and challenges. Human Molecular Genetics, 2014, 23(R1), R40-R46).

Although genetic scissors have been used as described above, there are limits depending on types of target cells (e.g., prokaryotic cells, eukaryotic cells, animal cells, and plant cells), and sites editable by the genetic scissors are also limited even in the same plant or animal, and research thereon is insufficient.

Under such backgrounds, the present inventors have found that a Cannabis gene may be efficiently and effectively edited by using a gene editing protein and verified, for the first time, that constructed Cannabis may grow to a plant body, thereby completing the present invention.

### DISCLOSURE

### Technical Problem

To solve the above technical problems, the present invention provides a composition for editing a Cannabis gene, a kit for editing a Cannabis gene, a kit for inhibiting Cannabis DNA expression and targeting the DNA, and a method of editing Cannabis DNA, each using a gene editing protein and gRNA; a method of preparing a gene-edited Cannabis including a step of forming calli; a gRNA for editing a Cannabis gene; and a use of the gRNA of the present invention and a use of both the gene editing protein and the gRNA for editing a Cannabis gene.

### Technical Solution

An object of the present invention is to provide a composition for editing a Cannabis gene using a gene editing protein.

Another object of the present invention is to provide a kit for editing a Cannabis gene using a gene editing protein.

Another object of the present invention is to provide a kit for inhibiting Cannabis DNA expression and targeting the DNA using a gene editing protein.

Another object of the present invention is to provide a method of editing Cannabis DNA using a gene editing protein.

Another object of the present invention is to provide a method of preparing a gene edited Cannabis including a step of forming calli.

Another object of the present invention is to provide a guide RNA (gRNA) or augmented RNA for editing a Cannabis gene.

Another object of the present invention is to provide a use of the gRNA of the present invention for editing a Cannabis gene.

Another object of the present invention is to provide a use of the gene editing protein and the gRNA of the present invention for editing a Cannabis gene.

### Advantageous Effects

The present invention has found, for the first time, that a Cannabis gene may be edited by using a gene editing protein, and a gene-edited Cannabis may be constructed efficiently and effectively by inducing calli thereafter.

### BRIEF DESCRIPTION OF DRAWINGS

FIG. 1 shows the protoplast isolation results by using Cannabis cotyledons.

FIG. 2 shows positions for selecting targets for Cas12f and Cpf1 in a THCA synthase gene (SEQ ID NO: 25).

FIGS. 3 and 4 show results of evaluation on THCAS gene indel generation ability by using gRNAs and Cpf1.

FIGS. 5 and 6 show results of evaluation on THCAS gene indel generation ability by using gRNAs and Cas12f.

FIG. 7 shows positions for selecting targets for Cas12f and Cpf1 in a PDS gene (SEQ ID NO: 26).

FIG. 8 shows results of evaluation on PDS gene indel generation ability by using gRNAs and Cpf1 or Cas9.

FIGS. 9 and 10 show results of evaluation on THCAS gene indel generation ability by using gRNAs and Cas12f.

FIG. 11 shows positions for selecting targets for Cpf1 in a CBDAS gene (SEQ ID NO: 26) and targets therefor.

FIG. 12 shows results of evaluation on CBDAS gene indel generation ability by using gRNAs and Cpf1.

FIGS. 13 and 14 show results of evaluation on CBDAS, THCAS, and CBDAS gene indel generation ability using gRNAs and Cpf1.

FIG. 15 shows THCAS and CBCAS Indel patterns by Cas12a.

FIG. 16 shows CBDAS Indel patterns by Cas12a.

FIGS. 17 and 18 show transfection results of protoplasts.

FIG. 19 shows minicalli induction results.

FIG. 20 shows a process of embryogenic callus formation and Cannabis redifferentiation.

FIG. 21 shows a process of redifferentiation of Cannabis from cell culture of Cannabis cells.

FIG. 22 shows wild-type guide RNAs for TnpB and modification sites (MS) MS1 to MS5 for engineered guide RNAs (augment RNAs) provided in the present invention.

FIG. 23 shows an exemplary structure indicating various modification sites for constructing engineered guide RNAs (gRNAs) of the present invention.

FIG. 24 shows an exemplary structure indicating a representative sequence of the augment RNA of the present invention.

### BEST MODE

Hereinafter, the contents of the present invention will be described in detail. On the other hand, the descriptions and the embodiments of one aspect disclosed in this application may be applied to descriptions and embodiments of other aspects with respect to common items. Also, all combinations of various elements disclosed in the present invention fall within the scope of the present invention. In addition, documents described in the present invention may be incorporated herein by reference. In addition, the scope of the present invention is not limited by the specific description described below.

An aspect of the present invention provides a composition for editing a Cannabis gene including a gene editing protein and a guide RNA (gRNA) and/or an augment RNA.

In the present invention, gene editing may be used interchangeably with gene correction, and the composition for gene editing may be used as a system for gene editing.

As used herein, the term “gene editing protein” refers to a protein utilized for gene editing and may be used interchangeably with endonuclease.

In an embodiment, the gene editing protein may be Cas or TnpB, or a functional analogue or variant thereof, but any naturally occurring, modified, and derived forms thereof may be included in the gene editing protein without limitation as long as they are utilized in gene editing techniques.

As used herein, the term “Cas” refers to a protein utilized in gene editing. A Cas protein used herein includes any proteins without limitation as long as the proteins are utilized in gene editing technology in the art. The protein utilized in the gene editing technology may recognize a guide RNA and cleave a target DNA/RNA, and specifically, may be Cas9, Cas12a (identical to Cpf1), Cas12b, Cas13 (including at least one of the Cas13 family, e.g., Cas13a, Cas13b, Cas13c, and Cas13d), Cas12f, or a functional analogue or variant thereof, but any naturally occurring, modified, and derived forms thereof may be included in the Cas protein without limitation as long as they are utilized in gene editing technology.

In an embodiment, the Cas may include at least one selected from the group consisting of Cas12f, Cas9, and Cpf1, but is not limited thereto.

As used herein, the term “Cas12f” is a small Cas protein classified as a type-V CRISPR nuclease identified in archaea, and the like. Like other Cas proteins, Cas12f also functions in combination with gRNA.

In an embodiment, the Cas12f of the present invention may be a naturally occurring Cas12f protein or a functional analogue or variant thereof, but is not limited thereto.

As used herein, the term “TnpB” is a protein conventionally known as a transposase. To date, the TnpB protein is only known as a transposon-encoded nuclease, and it is not known whether the TnpB protein has Cas endonuclease activity. In addition, a guide RNA for the TnpB protein is not known. The present invention is significant in the sense that the Cannabis gene was edited by using TnpB as an endonuclease for the first time.

In an embodiment, the TnpB of the present invention may be a naturally occurring TnpB protein or a functional analogue or variant thereof, but is not limited thereto. In addition, in the present invention, TnpB may also be referred to as CWCas12f1.

The present invention includes contents of Korean Patent Application No. 10-2021-0132306 by reference.

In an embodiment, the TnpB protein may include or consist of an amino acid sequence of SEQ ID NO: 27, the functional analogue may consist of an amino acid sequence in which 1 to 28 amino acids are removed or substituted from the N-terminus of the TnpB protein or an amino acid sequence in which 1 to 600 amino acids are added to the N-terminus or the C-terminus of the TnpB protein, wherein the functional analogue is characterized by not being a Cas12f1 protein consisting of an amino acid sequence of SEQ ID NO: 31, but is not limited thereto.

In an embodiment, the functional analogue may be characterized by consisting of at least one amino acid sequence selected from SEQ ID NOS: 28 to 30, but is not limited thereto.

In an embodiment, the functional analogue may be one prepared by further adding at least one amino acid sequence to the Cas12f1 protein consisting of the amino acid sequence of SEQ ID NO: 31, but is not limited thereto.

In an embodiment, because the gene editing protein and gRNA function in the form of a ribonucleoprotein (RNP) complex that is a protein: RNA complex, the composition for editing a Cannabis gene may include a gene editing protein and a gRNA in the form of a protein: RNA complex in the present invention.

In an embodiment, the Cas12f protein of the present invention may include or consist of an amino acid sequence of SEQ ID NO: 31, the Cas9 protein of the present invention may include or consist of an amino acid sequence of SEQ ID NO: 44, and the Cpf1 (identical to Cas12a) protein of the present invention may include or consist of an amino acid sequence of SEQ ID NO: 45, without being limited thereto.

In the present invention, although the expression ‘a polypeptide or protein including an amino acid sequence as set forth in a predetermined SEQ ID NO:’, “a polypeptide or protein consisting of an amino acid sequence as set forth in a predetermined SEQ ID NO:′ or “a polypeptide or protein having an amino acid sequence as set forth in a predetermined SEQ ID NO:′ is used, it is obvious that any protein including deletion, modification, substitution, conservative substitution, or addition of one or several amino acids may be used in the present invention as long as the protein has activity identical or equivalent to that of the polypeptide or protein consisting of the amino acid sequence of the SEQ ID NO:. For example, addition of a sequence not changing the function of the protein to the N-terminus and/or the C-terminus of the amino acid sequence, a naturally occurring mutation, a silent mutation thereof, or a conservative substitution thereof may be used.

The “conservative substitution” refers to substitution of one amino acid with another amino acid having a similar structural and/or chemical property. Such amino acid substitution may generally occur based on similarity of polarity, charge, solubility, hydrophobicity, hydrophilicity, and/or amphipathic nature of residues. In general, conservative substitution may have little effect or no effect on activity of a protein.

As used herein, the term “guide RNA (gRNA)” refers to an RNA that plays a role in cleaving of a target gene in a sequence-specific manner by inducing DNA sequence specificity of a target region. The guide RNAs (gRNAs) may be classified into single guide RNAs (sgRNAs) and dual guide RNAs (dual gRNAs) according to the number of RNAs constituting the gRNAs, but are not limited thereto.

In an embodiment, the gRNA of the present invention may be a sgRNA consisting of one RNA or a crRNA, but the types thereof are not limited as long as the gRNA may be used with a gene editing protein, and may consist of a modified nucleotide and/or a non-modified nucleotide. The modified nucleotide may include at least one selected from the group consisting of 2′-O-methyl RNA (2′-OMe RNA), 2′-O-methoxyethyl RNA (2′-MOE RNA), 2′-fluoro RNA (2′-F RNA), phosphorothioate RNA (PS RNA), 2′-amino RNA (2′—NH₂ RNA), 2′-fluoro arabinose nucleic acid (FANA), 4′-thio RNA (4′-S RNA), locked nucleic acid (LNA), threose nucleic acid (TNA), phosphorothioate DNA (PS DNA), DNA, peptide nucleic acid (PNA), phosphorodiamidate morpholino oligonucleotide (PMO), hexitol nucleic acid (HNA), cyclohexene nucleic acid (CeNA), and arabinose nucleic acid (ANA).

In an embodiment, the gRNA may be engineered, but is not limited thereto.

In an embodiment, the engineered gRNA may be one constituting a gene editing system by forming a complex with TnpB or a functional analogue thereof, but is not limited thereto. The present invention includes Korean Patent Application No. 10-2021-0132306 by reference.

In an embodiment, the engineered gRNA may be a gRNA engineered to have one or more nucleotides deleted, substituted, or added compared to the corresponding wild-type gRNA consisting of a nucleotide sequence of SEQ ID NO: 32, and a spacer region of the gRNA is characterized by having a nucleotide sequence consisting of 15 or more but not more than 50 nucleotides, but is not limited thereto.

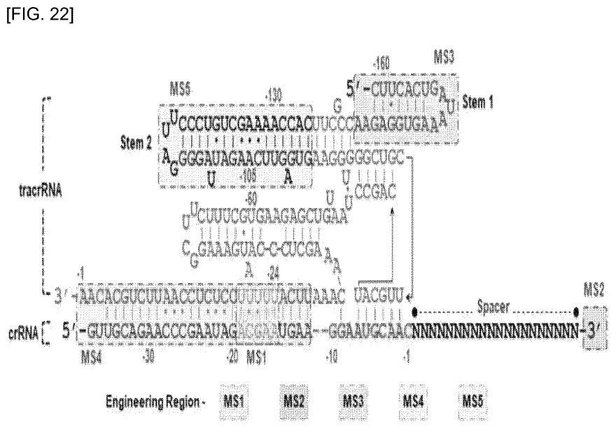

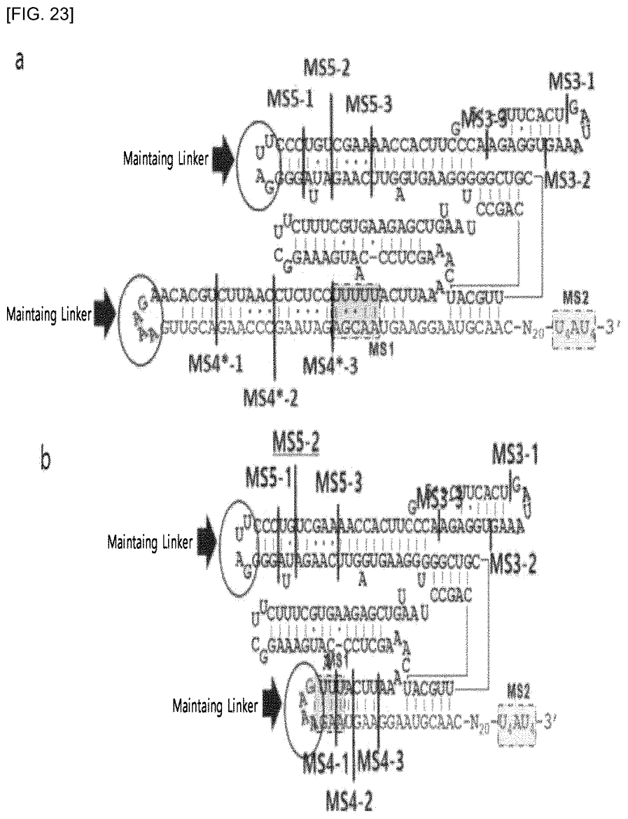

In an embodiment, the engineered gRNA may be modified as shown in FIGS. 22 and 23.

In an embodiment, the engineered gRNA may include an engineered transactivating CRISPR RNA (tracrRNA) or an engineered CRISPR RNA (crRNA), wherein the engineered tracrRNA may be modified not to contain 5 or more consecutive uridines in the sequence and to have a shorter nucleotide sequence than the wild-type tracrRNA, and the engineered crRNA may be characterized by including SEQ ID NO: 33 or a part of the sequence, without being limited thereto.

In an embodiment, the addition in the present invention may be characterized by a U-rich tail sequence added to the 3′-terminus of crRNA, but is not limited thereto.

In an embodiment, the gRNA of the present invention may consist of tracrRNA and crRNA.

In an embodiment of the above-described embodiments, the gRNA may include an engineered crRNA, but is not limited thereto.

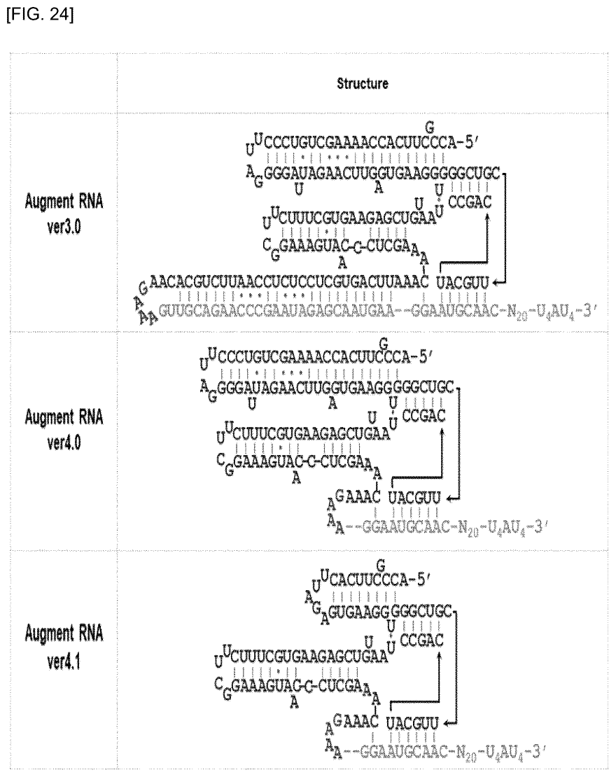

In an embodiment, the gRNA may be one shown in FIG. 24 and/or in SEQ ID NOS: 34 to 36, but is not limited thereto.

In an embodiment, the engineered crRNA may be prepared for the CRISPR/Cas12f1 system, but is not limited thereto. The present invention includes contents of Korean Patent Application Nos. 10-2021-0050093, 10-2021-0044152, and 10-2021-0051552 by reference.

In an embodiment, the Cannabis gene, as a target for gene editing in the present invention may be at least one of a Cannabis gene encoding tetrahydrocannabinolic acid synthase (THCAS), a Cannabis gene encoding phytoene desaturase (PDS), a Cannabis gene encoding Cannabidiolic acid synthase (CBDAS), and a Cannabis gene encoding Cannabichromenic acid synthase (CBCAS), but is not limited thereto.

Particularly, Cannabis is known to contain both tetrahydrocannabinol (THC) that is a drug component and cannabidiol (CBD) that is a medical component. In addition, regulations on Cannabis are currently being relaxed in the United States and Cannabis is becoming legal in many states of the United States, and accordingly, a huge market called Green Rush is formed and it is necessary to understand various components thereof. Moreover, imports and production of all kinds of Cannabis are prohibited in Korea due to regulations on Cannabis itself, and Cannabis components for medical purposes are also entirely dependent on imports. By applying the latest technology using genetic scissors according to an embodiment of the present invention, activity of precursors of active ingredients of Cannabis may be adjusted using the gene editing technology for editing genes of Cannabis (e.g., THCAS and/or PDS genes), so that various components such as medical components or commercial components may be adjusted, or the drug components may be removed.

When the target of gene editing is Cannabis, the gRNA may be a gRNA for editing Cannabis genes including at least one sequence selected from the group consisting of SEQ ID NOS: 1 to 14 and SEQ ID NOS: 38 to 42 as a target sequence, but is not limited thereto. A gRNA including at least one of SEQ ID NOS: 1 to 5 as a target sequence may be a gRNA for editing a Cannabis gene encoding tetrahydrocannabinolic acid synthase (THCAS), a gRNA including at least one of SEQ ID NOS: 6 to 14 as a target sequence may be a gRNA for editing a Cannabis gene encoding phytoene desaturase (PDS), a gRNA including at least one of SEQ ID NOS: 38 to 42 as a target sequence may be a gRNA for editing a Cannabis gene encoding cannabidiolic acid synthase (CBDAS), and a gRNA including SEQ ID NO: 5 as a target sequence may be a gRNA for editing not only a Cannabis gene encoding THCAS but also a Cannabis gene encoding cannabichromenic acid synthase (CBCAS).

In an embodiment, crRNAs (gRNAs) respectively targeting SEQ ID NOS: 1 to 5 and constructed by using a Cpf1 crRNA scaffold (SEQ ID NO: 50) may include, have, or consist of SEQ ID NOS: 51 to 55, respectively, and gRNAs respectively targeting SEQ ID NOS: 1 to 5 and constructed by using a Cas12f gRNA scaffold (SEQ ID NO: 56) may include, have, or consist of SEQ ID NOS: 57 to 61, respectively.

In an embodiment, crRNAs (gRNAs) respectively targeting SEQ ID NOS: 6 to 13 and constructed by using a Cpf1 crRNA scaffold (SEQ ID NO: 50) may include, have, or consist of SEQ ID NOS: 62 to 69, respectively, gRNAs respectively targeting SEQ ID NOS: 6 to 13 and constructed by using a Cas12f gRNA scaffold (SEQ ID NO: 56) may include, have, or consist of SEQ ID NOS: 70 to 77, respectively, and a gRNA targeting SEQ ID NO: 14 and constructed by using a Cas9 gRNA scaffold (SEQ ID NO: 83) may include, have, or consist of SEQ ID NO: 84.

In an embodiment, gRNAs respectively targeting SEQ ID NOS: 38 to 42 and constructed by using a Cpf1 (identical to Cas12a) crRNA scaffold (SEQ ID NO: 50) may include, have, or consist of SEQ ID NOS: 78 to 82, respectively. In an embodiment of the above-described embodiments, the gRNA of the present invention including at least one sequence of SEQ ID NOS: 1 to 14 and SEQ ID NOS: 38 to 42 as a target sequence may include at least one of SEQ ID NOS: 51 to 55 and SEQ ID NOS: 57 to 82.

In an embodiment of the above-described embodiments, the gRNA of the present invention may include at least one of SEQ ID NOS: 51 to 55 and SEQ ID NOS: 57 to 82.

The gRNA according to the present invention has excellent efficiency in Cannabis gene editing, particularly, gene editing using a gene editing protein (e.g., Cas and TnpB).

The gRNA including at least one of SEQ ID NOS: 1 to 14 and SEQ ID NOS: 38 to 42 as a target sequence may also be used with other endonucleases as well as Cas.

In an embodiment, a protein: gRNA complex for editing a Cannabis gene including a gRNA including at least one of SEQ ID NOS: 1 to 14 and SEQ ID NOS: 38 to 42 as a target sequence and an endonuclease; a composition for editing a Cannabis gene including the protein: gRNA complex; a kit for inhibiting Cannabis DNA expression and targeting the DNA including DNA encoding the gRNA including at least one of SEQ ID NOS: 1 to 14 and SEQ ID NOS: 38 to 42 as a target sequence, and an endonuclease or a polynucleotide encoding the same; and a kit for editing a Cannabis gene including DNA encoding the gRNA including at least one of SEQ ID NOS: 1 to 14 and SEQ ID NOS: 38 to 42 as a target sequence, and an endonuclease or a polynucleotide encoding the same may also be included in the present invention as long as the gRNA including at least one of SEQ ID NOS: 1 to 14 and SEQ ID NOS: 38 to 42 as a target sequence is used. The endonuclease may be any protein having gene cleavage ability without limitation, or may be selected from the group consisting of TnpB, Cpf1, Cas12f, and Cas9.

Although the gRNA is expressed by a predetermined SEQ ID NO: in the present invention, any sequence including addition, deletion, or substitution of a meaningless sequence may be included within the scope of the present invention as long as the use for gene editing is achieved.

In the present invention, the composition for editing a Cannabis gene is not limited in operational principles or forms thereof as long as the Cannabis gene may be edited by targeting the Cannabis gene. In addition, the composition of the present invention may further include, but is not limited to, one or more other components, solutions, and/or devices suitable for inhibiting gene expression and/or targeting genes. Examples of the other components may include an appropriate carrier, solubilizer, buffer, stabilizer, and the like, but are not limited thereto. The carrier may be a soluble carrier or an insoluble carrier, and examples of the soluble carrier may include a physiologically acceptable buffer known in the art, e.g., PBS, and examples of the insoluble carrier may include polystyrene, polyethylene, polypropylene, polyester, polyacrylonitrile, a fluorine resin, crosslinked dextran, polysaccharide, a polymer such as magnetic particles made by plating metal on latex, other paper, glass, metal, agarose, and any combination thereof, but are not limited thereto.

Another aspect of the present invention provides a kit for editing a Cannabis gene including: a gene editing protein or a polynucleotide encoding the same; and a gRNA or DNA encoding the gRNA.

The gene editing protein, gRNA, and the like are as described in other aspects.

The kit is not limited in operational principles or forms thereof as long as expression of DNA and/or RNA may be inhibited and DNA and/or RNA may be targeted thereby. In addition, the kit of the present invention may further include one or more other components, compositions, solutions, or devices suitable for inhibition of DNA and/or RNA expression and targeting DNA and/or RNA, but is not limited thereto. Examples of the other components may include an appropriate carrier, solubilizer, buffer, stabilizer, and the like, but are not limited thereto. The carrier may be a soluble carrier or an insoluble carrier, and examples of the soluble carrier may include a physiologically acceptable buffer known in the art, e.g., PBS, and examples of the insoluble carrier may include polystyrene, polyethylene, polypropylene, polyester, polyacrylonitrile, a fluorine resin, crosslinked dextran, polysaccharide, a polymer such as magnetic particles made by plating metal on latex, other paper, glass, metal, agarose, and any combination thereof, but are not limited thereto.

In addition, the kit of the present invention may further include guidelines for a user describing optimal conditions for reaction. The guidelines are printed materials that explain how to use the kit, for example, methods for preparing a buffer, suggested conditions for reaction, and the like. The guidelines include booklets in the form of pamphlets or leaflets, labels attached to the kit, and instructions on the surface of a package containing the kit. In addition, the guidelines includes information published or provided through electronic media such as the Internet.

Another aspect of the present invention provides a kit for inhibiting Cannabis DNA expression and targeting the DNA including: a gene editing protein or a polynucleotide encoding the same; and a gRNA or DNA encoding the gRNA.

The gene editing protein, gRNA, kit, and the like are as described in other aspects.

Another aspect of the present invention provides a method of editing Cannabis DNA, the method including treating the Cannabis plant body with the composition.

Cells, tissues, protoplasts, and the like may be treated with the composition, without being limited thereto.

Another aspect of the present invention provides a method of editing Cannabis DNA, the method including culturing Cannabis protoplasts including a gene editing protein and a gRNA.

As used herein, the term “culture” refers to growing the Cannabis protoplasts under appropriately adjusted environmental conditions. A culture process of the present invention may be performed according to appropriate culture media and culture conditions known in the art. Such a culture process may be adjusted by one of ordinary skill in the art according to a selected strain. Specifically, the culture process may be, but is not limited to, batch culture, continuous culture, and fed-batch culture. Although the culture medium used to culture the Cannabis protoplasts and other culture conditions according to the present invention are not particularly limited as long as the culture medium is commonly used for culturing Cannabis protoplasts, the Cannabis protoplasts of the present invention may be cultured in a common culture medium containing carbon sources, nitrogen sources, phosphorus sources, inorganic compounds, amino acids, and/or vitamins under aerobic conditions while adjusting temperature, pH, and the like.

Provided is a method of editing Cannabis DNA, the method including culturing Cannabis protoplasts including an endonuclease (e.g., at least one selected from the group consisting of TnpB, Cpf1, Cas12f, and Cas9) and a gRNA.

Regardless of types of the gRNA, in view of the objects of the present invention, the culturing is not limited as long as gene editing is performed using an endonuclease protein and a gRNA by the culturing in Cannabis protoplasts containing the endonuclease protein and the gRNA.

In an embodiment, the protoplast may be derived from Cannabis leaf mesophyll, but is not limited thereto.

In an embodiment, the modifying Cannabis DNA, targeting DNA, and editing DNA may be adjusting components of Cannabis (components contained in Cannabis). For example, adjusting the components of Cannabis such as decreases in drug components (e.g., THCA) and/or increases in medical components may be included therein.

The gene editing protein, gRNA, and the like are as described above in other aspects.

Another aspect of the present invention provides a method of preparing Cannabis. Specifically, the method of preparing Cannabis includes: culturing protoplasts isolated from Cannabis; forming calli in the protoplasts; forming somatic embryos in the calli; and differentiating the somatic embryos into plant bodies or mature plants.

In an embodiment, the protoplast may include a gene editing protein and a gRNA and may be one from which drug components were removed by gene editing using the gene editing protein and the gRNA.

In an embodiment of the above-described embodiments, the method of preparing Cannabis of the present invention may be a method of preparing a gene-edited Cannabis.

Another aspect of the present invention provides a guide RNA (gRNA) for editing a Cannabis gene.

To control various Cannabis components such as medical components or commercial components by adjusting activity of precursors of active ingredients by using the gRNA of the present invention, the Cannabis gene may include at least one of a gene encoding tetrahydrocannabinolic acid synthase (THCAS) and a gene encoding phytoene desaturase (PDS).

In an embodiment, the gRNA for editing a Cannabis gene may include one of SEQ ID NOS: 1 to 14 and SEQ ID NOS: 38 to 42 as a target sequence, but is not limited thereto. The gRNA including at least one of SEQ ID NOS: 1 to 5 as a target sequence may be a gRNA for editing a Cannabis gene encoding tetrahydrocannabinolic acid synthase (THCAS), the gRNA including at least one of SEQ ID NOS: 6 to 14 as a target sequence may be a gRNA for editing a Cannabis gene encoding phytoene desaturase (PDS), the gRNA including at least one of SEQ ID NOS: 38 to 42 as a target sequence may be a gRNA for editing a Cannabis gene encoding cannabidiolic acid synthase (CBDAS), and the gRNA including SEQ ID NO: 5 as a target sequence may be a gRNA for editing not only a Cannabis gene encoding THCAS but also a Cannabis gene encoding cannabichromenic acid synthase (CBCAS).

The gRNA is as described above in other aspects.

Another aspect of the present invention provides a use of the guide RNA (gRNA) of the present invention for editing a Cannabis gene.

Another aspect of the present invention provides a use of the gene editing protein and the guide RNA (gRNA) of the present invention for editing a Cannabis gene.

The gene editing protein, gRNA, and Cannabis gene are as described above in other aspects.

### [Mode of Disclosure]

Hereinafter, the present invention will be described in more detail with reference to the following examples. However, the following examples experimental examples are merely presented to exemplify the present invention, and the scope of the present invention is not limited thereto.

### Example 1: Preparation of Cannabis Gene Editing

1-1: Isolation of Protoplasts from Cannabis Leaf Mesophyll

After sterilization, Cannabis was seeded in a ½ MS medium (containing 2.2 g of ½ MS salts and organics (Duchefa), Vit. 15 g of sucrose, and 8 g of agar) per 1 L at 25° C. under light conditions for 16 hours/dark conditions for 8 hours. 1 to 2 weeks later, the first true leaves or cotyledons of the Cannabis were used.

Subsequently, 10 ml of an enzyme solution (containing 20 mM MES (pH 5.7), 20 mM KCl, 10 mM CaCl₂), 0.1% BSA, 1% Viscozyme, 0.5% Celluclast, and 0.5% pectinase) was filtered by a syringe and added to a plate, and the leaves were cut at intervals from 1 to 2 mm and added to the solution. The prepared solution was allowed to stand in a shaking incubator under dark conditions at 50 rpm, at 25° C., for 5 to 7 hours.

After filtering protoplasts by using a nylon mesh, 5 to 6 ml of W5 (containing 154 mM NaCl, 125 mM CaCl₂), 5 mM KCl, and 2 mM MES, pH 5.7) was added to the remaining protoplasts, and the resultant was filtered by using the nylon mesh and collected in one place. Then, centrifugation was performed at 610 rpm for 5 minutes, and a supernatant was removed therefrom.

After 2 ml of W5 was added to the contents from which the supernatant was removed and the contents were carefully dispersed therein, 8 ml of W5 was further added thereto, followed by centrifugation at 610 rpm for 5 minutes. This step was repeated one more time.

In addition, the resultant was dispersed in 7 ml of an MMG solution (containing 0.4 M mannitol, 4 mM MES (pH 5.7), and 15 mM MgCl₂), followed by centrifugation at 610 rpm for 5 minutes, and this process was repeated one more time. In the next step, after carefully resuspending the resultant in 2 ml to 3 ml of the MMG solution, 20 μl was isolated therefrom and cells contained therein were counted by using a hemocytometer. A final concentration was adjusted approximately to 1 to 3×10⁶ cells/ml.

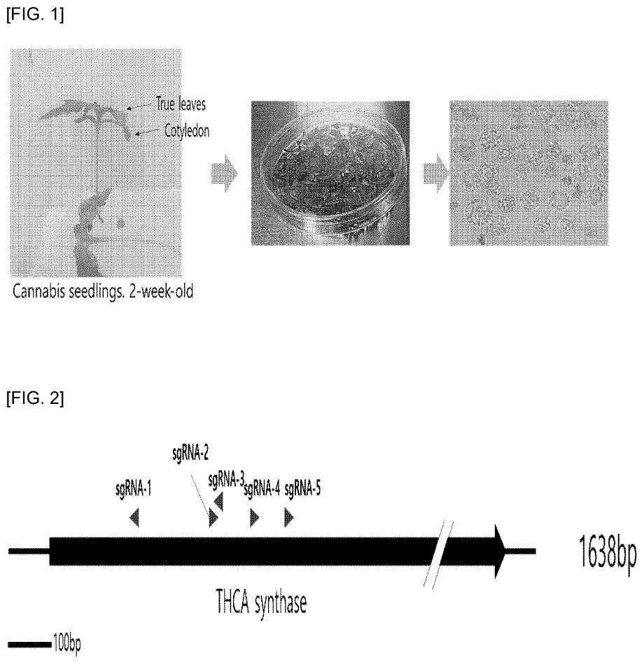

Results of protoplast isolation using Cannabis cotyledons are shown in FIG. 1.

1-2: Construction of Cas12f gRNA

After synthesizing forward oligonucleotides (SEQ ID NO: 15: 5′-atacgactcactatagACCGCTTCACttagAGTGAAGGTGGGCTGCTTGCATCAGCCT AATGTCGAGAAGTGCTTTCTTCGGAAAGTAACCCTCGAAACAAA-3′) and reverse oligonucleotides (SEQ ID NO: 16: 5′-reverse complement target sequence+GTTGCATTCCTTTCTTTGTTTCGAGGGTTACTTTCCG-3′) of a T7+tracrRNA sequence, 1 μl of the 100 UM forward oligonucleotides, 1 μl of the 100 UM reverse oligonucleotides, and 8 μl of DW were mixed to a total volume of 10 μl, and the mixture was allowed to stand in a PCR machine at 95° C. for 5 minutes; at 55° C. for 10 minutes; and at 4° C. for annealing of a complementary region of 42 bp.

2 μl of dNTPs (10 mM), 2 μl of a 10X NEB 2.1 buffer, 0.5 μl of a T4 DNA polymerase, and 5 μl of DW were added thereto, and the mixture was allowed to stand at 12° C. for 20 minutes; and at 4° C. to prepare double strand DNA.

After column purification, amplification was performed by PCR using the following primers.

Forward (SEQ ID NO: 17):
5′-gaaaTtaatacgactcactatagACCG-3
Reverse (SEQ ID NOS: 18 and 19):
5′-AAAAAATAAAA reverse-complement
target sequence-GTTGCATTCCtttcT
TTGTTT-3′

After PCR purification, in-vitro transcription was performed. 4 to 6 μg of a PCR template, 2 μl of 1 M DTT, and a total of 32 μl of 100 mM NTPs (8 μl of each of ATP, GTP, CTP, and UTP), 14 μl of 0.2 M MgCl₂, 20 μl of a 10X T7 RNA polymerase buffer, 1 μl of a T7 RNA polymerase (high conc.), 5 μl of an RNase inhibitor (NEB), and DW were mixed to a total volume of 200 μl, followed by incubation at 37° C. O/N.

Then, 23 μl of a 10X DNase I buffer and 10 μl of DNase I were added thereto, followed by incubation at 37° C. for 1 hour and then purification with a Monarch RNA cleanup kit. The resultant was eluted with 50 to 100 μl to obtain a guide RNA.

1-3: Construction of Cas9 gRNA

After synthesizing reverse oligonucleotides (SEQ ID NO: 20: 5′-aGCACCGACTCGGTGCCACTTTTTCAAGTTGATAACGGACTAGCCTTATTTTA ACTTGCTATTTCTAGCTCTAAAAC-3′) and forward oligonucleotides (SEQ ID NO: 21 and 22: 5′-AATACGACTCACTATAGG+target sequence+GTTTTAGAGCTAGAAATAGCAAG-3′) of a gRNA scaffold sequence, 1 μl of 100 μM forward oligonucleotides, 1 μl of the 100 μM reverse oligonucleotides, and 8 μl of DW were mixed to a total volume of 10 μl, and the mixture was allowed to stand in a PCR machine at 95° C. for 5 minutes; at 55° C. for 10 minutes; and at 4° C. for annealing of a complementary region of 23 bp.

2 μl of dNTPs (10 mM), 2 μl of a 10X NEB 2.1 buffer, 0.5 μl of a T4 DNA polymerase, and 5 μl of DW were added thereto, and the mixture was allowed to stand at 12° C. for 20 minutes; and at 4° C. to prepare double strand DNA.

After column purification, amplification was performed by PCR using the following primers.

Forward (SEQ ID NO: 23):
5′-AATTTAATACGACTCACTATAGG-3
Reverse (SEQ ID NO: 24):
5′-aaaaGCACCGACTCGGT-3′

Processes after the in-vitro transcription were performed in the same manner as those of the construction of gRNA of Example 1-2.

1-4: Construction of Cas12a (Cpf1) gRNA

After synthesizing reverse oligonucleotides (5′-reverse complement target sequence (reverse complement of gRNA spacer sequence)+ATCTACAAGAGTAGAAATTA-3′; SEQ ID NO: 46) and forward oligonucleotides (5′-GAAATTAATACGACTCACTATAGTAATTTCTACTCTTGTAGAT-3′; SEQ ID NO: 47) of a gRNA scaffold sequence, 1 μl of the 100 UM forward oligonucleotides, 1 μl of the 100 UM reverse oligonucleotides, and 8 μl of DW were mixed to a total volume of 10 μl, and the mixture was allowed to stand in a PCR machine at 95° C. for 5 minutes; at 55° C. for 10 minutes; and at 4° C. for annealing of a complementary region of 20 bp.

2 μl of dNTPs (10 mM), 2 μl of a 10X NEB 2.1 buffer, 0.5 μl of a T4 DNA polymerase, and 5 μl of DW were added thereto, and the mixture was allowed to stand at 12° C. for 20 minutes; and at 4° C. to prepare double strand DNA.

After column purification, amplification was performed by PCR using the following primers.

Forward (SEQ ID NO: 48):
5′-GAAATTAATACGACTCACTATAG-3′
Reverse (SEQ ID NO: 49):
5′-AAAAAATAAAA + Reverse complement target
sequence-3′

Processes after the in-vitro transcription were performed in the same manner as those of the construction of gRNA of Example 1-2.

### Example 2: Genetic Modification of Cannabis Protoplast (1)—THCAS Knock-Out

2-1: Construction of gRNA for THCAS

By using the methods described in Examples 1-2 and 1-4, gRNAs for positions shown in FIG. 2 were constructed to select targets for Cas12f and Cpf1 in a THCA synthase gene of 1,638 bp (SEQ ID NO: 25). Table 1 below shows target sequences recognized by the gRNAs and including a PAM sequence and a target sequence.

TABLE 1

| SEQ ID | | |
|---|---|---|
| NO: | Name | sequence (5′→3′) |
| 1 | sgRNA-1 | TTTGTATTGTCGAATTCAGGATAG |
| 2 | sgRNA-2 | TTTGTTGTAGTAGACTTGAGAAAC |
| 3 | sgRNA-3 | TTTGATCGAATGCATGTTTCTCAAG |
| 4 | sgRNA-4 | TTTATTATTGGATCAATGAGAAG |
| 5 | sgRNA-5 | TTTAGTGGAGGAGGCTATGGAGC |

Accordingly, crRNAs (SEQ ID NOS: 51 to 55) respectively targeting SEQ ID NOS: 1 to 5 were constructed by using a Cpf1 crRNA scaffold (SEQ ID NO: 50), and gRNAs (SEQ ID NOS: 57 to 61) respectively targeting SEQ ID NOS: 1 to 5 were constructed by using a Cas12f gRNA scaffold (SEQ ID NO: 56).

2-2: Evaluation on THCAS Gene Cleavage Ability Using gRNA and Cpf1 (Endonuclease)

Indel evaluation was performed by deep sequencing (deep-seq). A fragment of about 250 bp containing a target sequence region, indel of which is desired to be identified, was amplified using a target sequence-specific primer additionally containing an Illumina adapter sequence at the 5′ region of the primer. After secondary PCR was performed using a primer containing an Illumina index sequence (Illumina TruSeq HT dual indexes), the resultant was subjected to PCR purification and sequencing using Illumina iSeq 100. Indel results were analyzed by using the MAUND program (https://github.com/ibs-cge/maund).

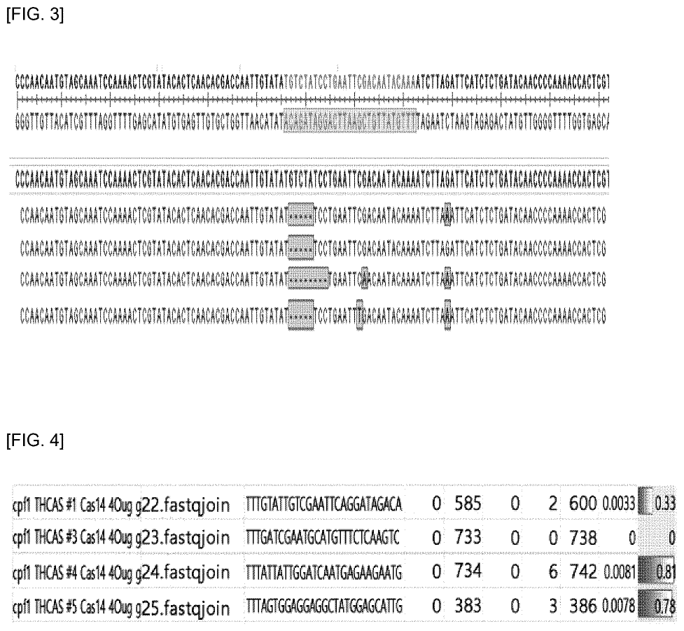

Indel evaluation results are shown in Table 2 below and FIGS. 3 and 4.

TABLE 2

| Endonuclease | gRNA target | Indel (%) |
|---|---|---|
| Cpf1 | sgRNA-1 | 0.33 |
| | sgRNA-3 | 0 |
| | sgRNA-4 | 0.81 |
| | sgRNA-5 | 0.78 |

As shown in Table 2, it was confirmed that the gRNAs targeting sgRNA-1, sgRNA-4, and sgRNA-5 had far superior target gene-specific cleavage ability.

2-3: Evaluation on THCAS Gene Cleavage Ability Using gRNA and Cas12f (Endonuclease)

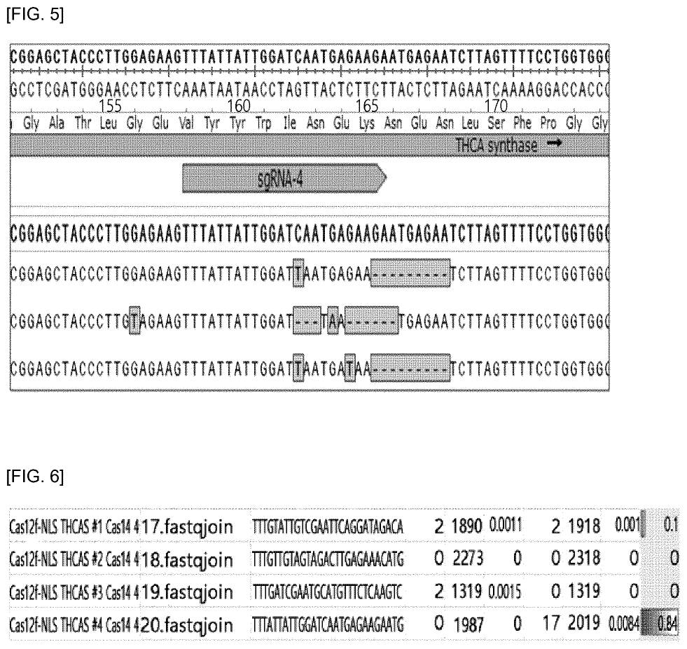

Indel evaluation results are shown in Table 3 below and FIGS. 5 and 6.

TABLE 3

| Endonuclease | gRNA target | Indel (%) |
|---|---|---|
| Cas12f | sgRNA-1 | 0.1 |
| | sgRNA-2 | 0 |
| | sgRNA-3 | 0 |
| | sgRNA-4 | 0.84 |

As shown in Table 3, it was confirmed that the gRNAs targeting sgRNA-1 and sgRNA-4 had far superior target gene-specific cleavage ability.

### Example 3: Genetic Modification of Cannabis Protoplast (2)—PDS Knock-Out

3-1: Construction of gRNA for PDS

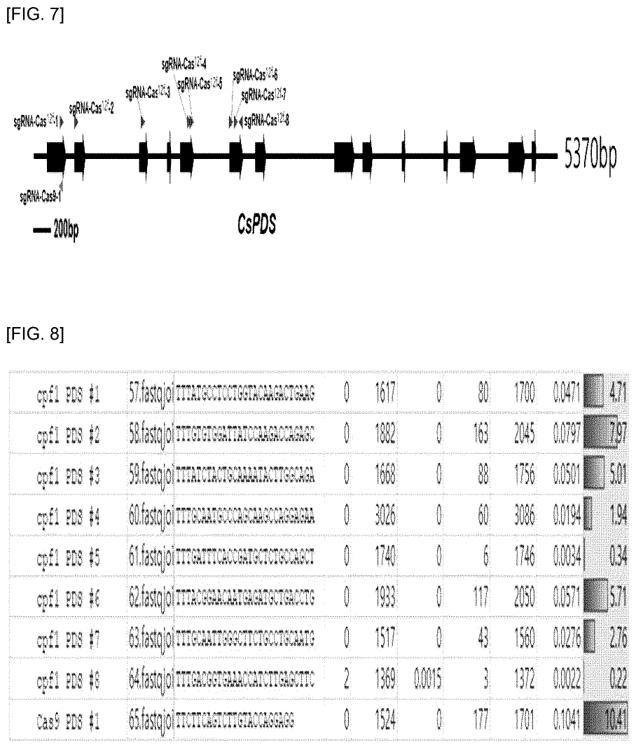

By using the methods described in Examples 1-2 and 1-4, gRNAs for positions shown in FIG. 7 were constructed to select targets for Cas12f, Cpf1, and Cas9 in a PDS gene of 5,370 bp (SEQ ID NO: 32). Table 4 below shows target sequences recognized by the gRNAs and including a PAM sequence and a target sequence.

TABLE 4

| SEQ ID | | |
|---|---|---|
| NO: | Name | Sequence (5′→3′) |
| 6 | sgRNA-1 | TTTATGCCTCCTGGTACAAGACTG |
| 7 | sgRNA-2 | TTTGTGTGGATTATCCAAGACCAG |
| 8 | sgRNA-3 | TTTATCTACTGCAAAATACTTGGC |
| 9 | sgRNA-4 | TTTGCAATGCCCAGCAAGCCAGGAG |
| 10 | sgRNA-5 | TTTGATTTCACCGATGCTCTGCCAG |
| 11 | sgRNA-6 | TTTACGGAACAATGAGATGCTGAC |
| 12 | sgRNA-7 | TTTGCAATTGGGCTTCTGCCTGCAATG |
| 13 | sgRNA-8 | TTTGACGGTGAAACCATCTTGAGC |
| 14 | sgRNA-9 | TTCTTCAGTCTTGTACCAGGAGG |

Accordingly, crRNAs (SEQ ID NOS: 62 to 69) respectively targeting SEQ ID NOS: 6 to 13 were constructed by using a Cpf1 crRNA scaffold (SEQ ID NO: 50), gRNAs (SEQ ID NOS: 70 to 77) respectively targeting SEQ ID NOS: 6 to 13 were constructed by using a Cas12f gRNA scaffold (SEQ ID NO: 56), and a gRNA (SEQ ID NO: 84) targeting SEQ ID NO: 14 was constructed by using a Cas9 gRNA scaffold (SEQ ID NO: 83).

3-2: Evaluation on PDS Gene Cleavage Ability Using gRNA and Cpf1 or Cas9 (Endonuclease)

Indel evaluation was performed by deep sequencing (deep-seq). A fragment of about 250 bp containing a target sequence region, indel of which is desired to be identified, was amplified using a target sequence-specific primer additionally containing an Illumina adapter sequence at the 5′ region of the primer. After secondary PCR was performed using a primer containing an Illumina index sequence (Illumina TruSeq HT dual indexes), the resultant was subjected to PCR purification and sequencing using Illumina iSeq 100. Indel results were analyzed by using the MAUND program (https://github.com/ibs-cge/maund).

Indel evaluation results are shown in Table 5 below and FIG. 8.

TABLE 5

| Endonuclease | gRNA target | Indel (%) |
|---|---|---|
| Cpf1 | sgRNA-1 | 4.71 |
| | sgRNA-2 | 7.97 |
| | sgRNA-3 | 5.01 |
| | sgRNA-4 | 1.94 |
| | sgRNA-5 | 0.34 |
| | sgRNA-6 | 5.71 |
| | sgRNA-7 | 2.76 |
| | sgRNA-8 | 0.22 |
| Cas9 | sgRNA-9 | 10.41 |

As shown in Table 5, it was confirmed that the gRNAs targeting Cpf1-sgRNA-1 to sgRNA-8 and Cas9-sgRNA-9 had far superior target gene-specific cleavage ability.

3-3: Evaluation on PDS Gene Cleavage Ability Using gRNA and Cas12f (Endonuclease)

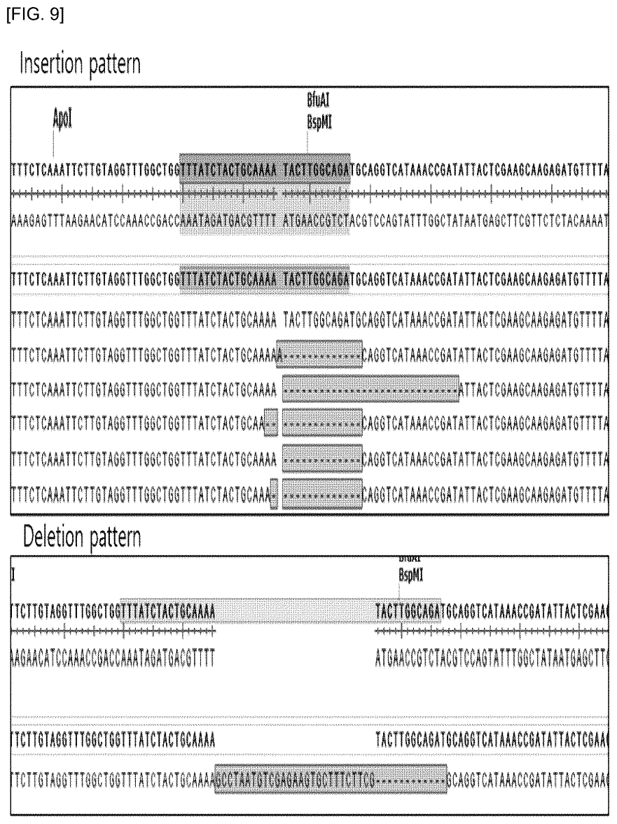

Indel evaluation results are shown in Table 6 below and FIGS. 9 and 10.

TABLE 6

| Endonuclease | gRNA target | Indel (%) |
|---|---|---|
| Cas12f | sgRNA-4 | 0.9 |

As shown in Table 6, it was confirmed that the gRNA targeting Cas12f-sgRNA-4 had far superior target gene-specific cleavage ability.

### Example 4: Genetic Modification of Cannabis Protoplast (3)—CBDAS Knock-Out

4-1: Construction of gRNA for CBDAS

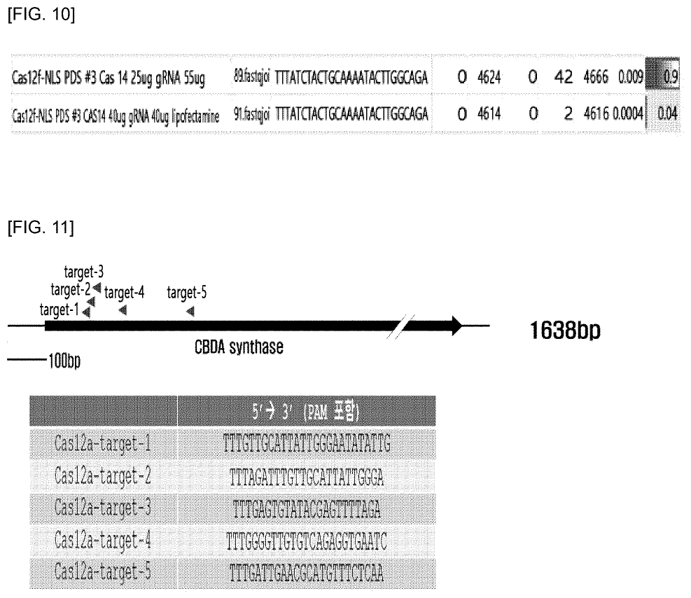

By using the method described in Example 1-4, gRNAs for positions shown in FIG. 11 were constructed to select targets in a CBDA synthase gene (SEQ ID NO: 37). Table 7 below shows gRNA target sequences including a PAM sequence and a target sequence.

TABLE 7

| | | SEQ |
|---|---|---|
| | | ID |
| | 5′→3′ | NO: |
| Cas12a-target-1 | TTTGTTGCATTATTGGGAATATATTG | 38 |
| Cas12a-target-2 | TTTAGATTTGTTGCATTATTGGGA | 39 |
| Cas12a-target-3 | TTTGAGTGTATACGAGTTTTAGA | 40 |
| Cas12a-target-4 | TTTGGGGTTGTGTCAGAGGTGAATC | 41 |
| Cas12a-target-5 | TTTGATTGAACGCATGTTTCTCAA | 42 |

Accordingly, gRNAs (SEQ ID NOS: 78 to 82) respectively targeting SEQ ID NOS: 38 to 42 were constructed using the Cpf1 (identical to Cas12a) crRNA scaffold (SEQ ID NO: 50).

4-2: Evaluation on CBDAS Gene Cleavage Ability Using gRNA and Cas12a (Cpf1; Endonuclease)

Indel evaluation was performed by deep sequencing (deep-seq). A fragment of about 250 bp containing a target sequence region, indel of which is desired to be identified, was amplified using a target sequence-specific primer additionally containing an Illumina adapter sequence at the 5′ region of the primer. After secondary PCR was performed using a primer containing an Illumina index sequence (Illumina TruSeq HT dual indexes), the resultant was subjected to PCR purification and sequencing using Illumina iSeq 100. Indel results were analyzed by using the MAUND program (https://github.com/ibs-cge/maund).

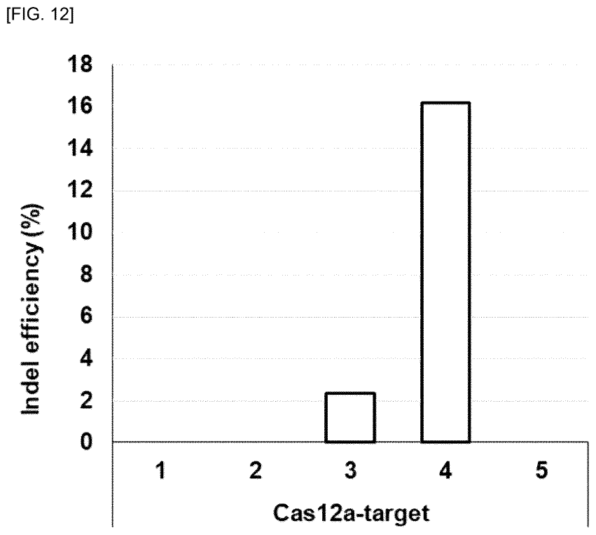

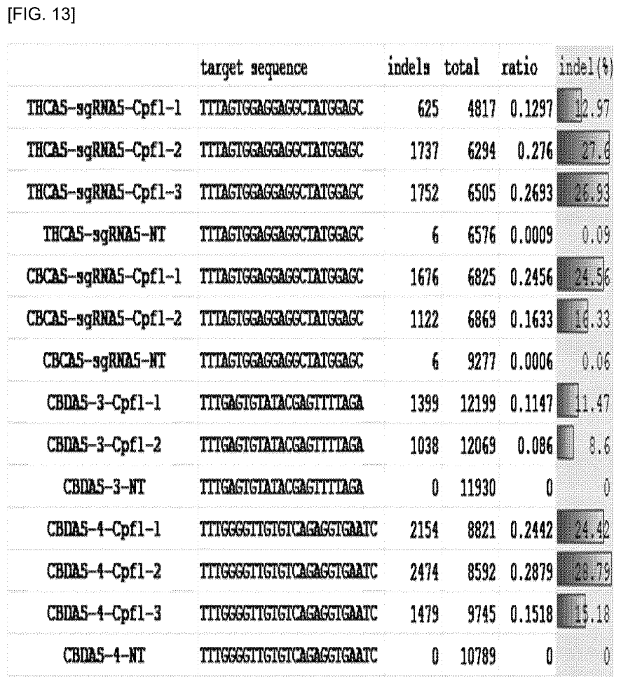

Indel evaluation results are shown in Table 8 and FIG. 12.

TABLE 8

| | PAM + target | Total | | Indel |
|---|---|---|---|---|
| | Sequence | read | Indel | (%) |
| Cas12a- | TTTGTTGCATTAT | 9353 | 0 | 0 |
| target-1 | TGGGAATATATTG | | | |
| Cas12a- | TTTAGATTTGTTG | 11920 | 0 | 0 |
| target-2 | CATTATTGGGA | | | |
| Cas12a- | TTTGAGTGTATAC | 10014 | 234 | 2.34 |
| target-3 | GAGTTTTAGA | | | |
| Cas12a- | TTTGGGGTTGTGT | 10120 | 1636 | 16.17 |
| target-4 | CAGAGGTGAATC | | | |
| Cas12a- | TTTGATTGAACGC | 8099 | 0 | 0 |
| target-5 | ATGTTTCTCAA | | | |

As shown in Table 8, it was confirmed that the gRNAs targeting target-3 and target-4 had far superior target gene-specific cleavage ability.

### Example 5: Evaluation on CBCAS, THCAS, and CBDAS Gene Cleavage Ability Using gRNA and Cas12a (Cpf1; Endonuclease)

Indel evaluation was performed by deep sequencing (deep-seq). A fragment of about 250 bp containing a target sequence region, indel of which is desired to be identified, was amplified using a target sequence-specific primer additionally containing an Illumina adapter sequence at the 5′ region of the primer. After secondary PCR was performed using a primer containing an Illumina index sequence (Illumina TruSeq HT dual indexes), the resultant was subjected to PCR purification and sequencing using Illumina iSeq 100. Indel results were analyzed by using the MAUND program (https://github.com/ibs-cge/maund).

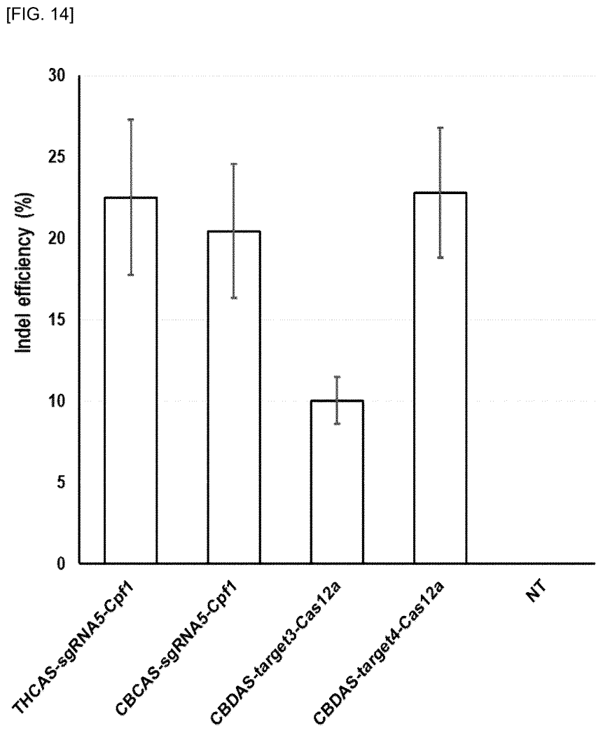

Indel evaluation results are shown in FIGS. 13 and 14. As shown in FIGS. 13 and 14, it was confirmed that the gRNA targeting sgRNA-5 of Table 1 by CAS12a was a THCAS targeting crRNA with high efficiency and also exhibited high efficiency in protoplasts by targeting the CBCAS gene (SEQ ID NO: 43). In addition, it was confirmed that CAS12a exhibited far superior CBDAS gene-specific cleavage ability for the target 3 and the target 4. THCAS and CBCAS

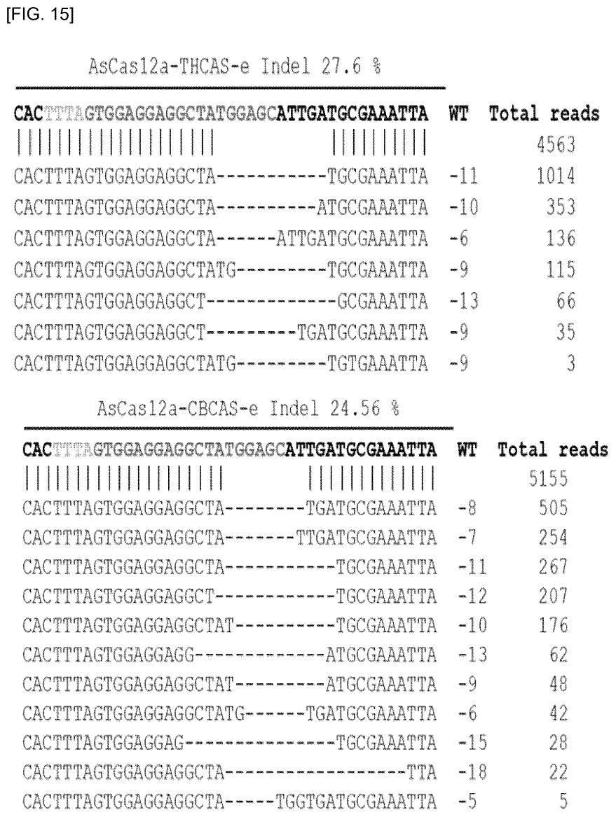

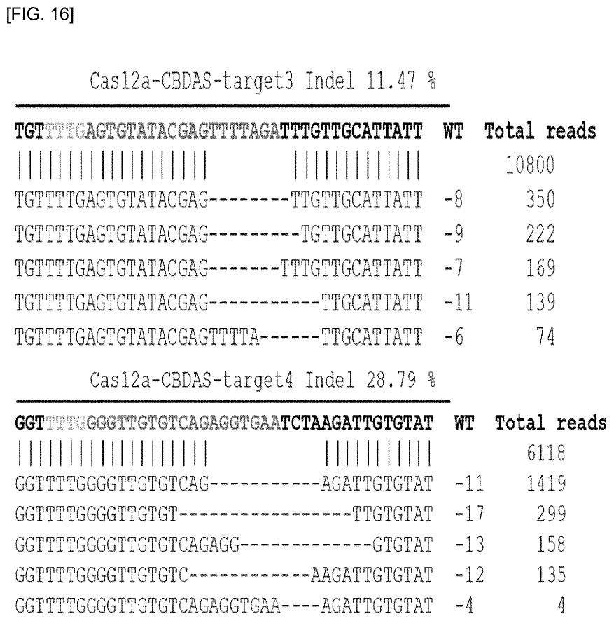

Indel patterns by Cas12a are shown in FIG. 15, and a CBDAS Indel pattern is shown in FIG. 16.

### Example 6: Protoplast Transfection

### 6-1: Protoplast Transfection

A PEG solution (containing 0.2 M mannitol, 100 mM CaCl₂), and 50% W/V PEG4000) was prepared on the day of use. Subsequently, an endonuclease protein and a gRNA were mixed in an appropriate ratio to a total volume of 20 μl and incubated in a waterbath at 37° C. for 15 minutes.

200 μl of protoplasts with an adjusted concentration were aliquoted into 2 ml tubes (2 ml round-bottom tubes). A total volume of 20 μl of a RNP complex (gRNA-endonuclease), mRNA, or plasmid for transfection was added to each cell and mixed in the tube.

Immediately after adding 200 μl of filtered 50% PEG thereto, the mixture was evenly mixed by pipetting. After 5 minutes, 500 μl of a W5 solution was added thereto and mixed by inverting twice or three times. In addition, after 5 minutes, 500 μl of the W5 solution was added thereto and mixed by inverting. Then, the resultant was subjected to centrifugation at 590 rpm for 5 minutes.

After removing the supernatant from the centrifuged solution, 1 ml of Medium E was added thereto and cell pellets were dispersed therein by inverting. In addition, the resultant was subjected to centrifugation at 590 rpm for 5 minutes. Then, the supernatant was removed therefrom, 1 ml of Medium E was added thereto, and cell pellets were dispersed therein by inverting.

1 ml of Medium E was added to 6-well plates whose bottom was coated with 5% BSA (filtered). 600 μl of protoplasts in Medium E was added to each well. After sealing with a micropore tape, the protoplasts were incubated at 25° C. under dark conditions. The composition of the medium are as follows.

### <Medium E>

Per 1 liter, 10 ml of macro stock, 1.25 ml of CaCl₂) stock (2.5 mM), 10 ml of Iron stock, 1 ml of micro stock, 5 ml of Vit. mix 1 stock, 5 ml of Vit. mix 2 stock, 5 ml of Vit. mix 3 stock, 20 ml of sugar stock, 10 ml of organic acid stock, 500 mg of casein hydrolysate, 33.7 g of glucose (0.17 M), 30.92 g of mannitol (0.17 M), 1 g of BSA, 1 mg of NAA, and 0.4 mg of BAP, pH 5.6 (KOH)

### <Macro Stock>

Per 1 liter, 74 g of KNO₃, 49.2 g of MgSO₄·7H₂O, and 3.4 g of KH₂PO₄

### <Iron Stock>

Per 100 ml, 140 mg of Na₂EDTA and 190 mg of FeSO₄·7H₂O

### <Micro Stock>

Per 100 ml, 150 mg of H₃BO₃, 500 mg of MnSO₄·H₂O, 100 mg of ZnSO₄·7H₂O, 12 mg of Na₂MoO₄·2H₂O, 1.2 mg of CuSO₄·5H₂O, 1.2 mg of CoCl₂·6H₂O, and 38 mg of KI

<Vit. Mix 1 Stock>

Per 100 ml, 50 mg of pantothenoic acid, 50 mg of choline chloride, 100 mg of ascorbic acid, 1 mg of p-aminobenzoic acid, 50 mg of nicotinic acid, 50 mg of pyridoxine-HCl, and 500 mg of thiamine-HCl

<Vit. Mix 2 Stock>

Per 100 ml, 20 mg of folic acid, 0.5 mg of biotin, and 1 mg of cyanocobalamin (Vit. B12)

<Vit. Mix 3 Stock>

Per 100 ml, 0.5 mg of cholecalciferol (Vit. D)

### <Sugar Stock>

Per 100 ml, 625 mg of sorbitol, 625 mg of sucrose, 625 mg of D(−)fructose, 625 mg of D(−)ribose, 625 mg of D(+)xylose, 625 mg of D(+)mannose, 625 mg of L(+)rhamnose monohydrate, 625 mg of D(+)cellobiose, and 250 mg of myo-inositol

### <Organic Acid Stock>

Per 100 ml, 100 mg of pyruvic acid, 200 mg of fumaric acid, 200 mg of citric acid monohydrate, and 200 mg of DL-malic acid

### 6-2: 35S: GFP Counting

The 35S: eGFP vector (Addgene plasmid #80127) was purchased and 30 μg of the plasmid was transfected using PEG (in the same manner as the method for protoplast RNP transfection), followed by incubation under dark conditions at 25° C. After about 12 hours, observation of GFP expression was available.

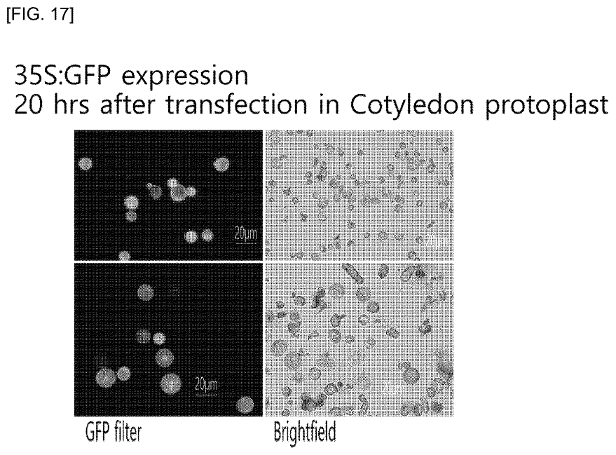

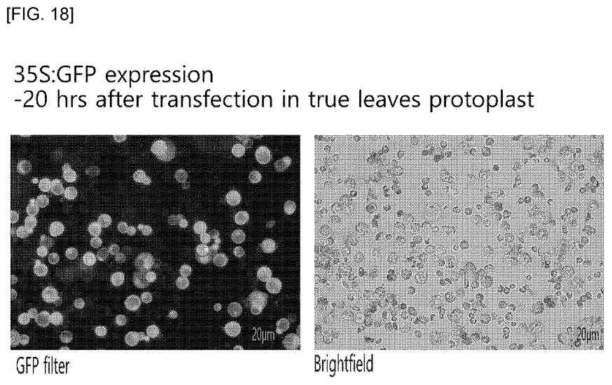

In the observation of GFP expression, living protoplasts were observed as intact circles in a brightfield channel, but chloroplasts emit red light in the case where GFP was not expressed in the GFP filter due to strong auto-fluorescence of chloroplasts. By applying these two conditions, the total number of protoplasts was measured, and transfection efficiency was evaluated in the protoplasts in which GFP was expressed by calculating an average from three or more pictures for measuring green fluorescent protoplasts in the GFP filter (FIGS. 17 and 18).

### Example 7: Callus Induction

After the protoplast transfection, the protoplasts in Medium E were mixed with an alginate solution (containing 2.8% w/v of alginic acid-Na salt and 0.4 M of sorbitol), and then the mixture was dropped on agar (setting agar; 0.4 M sorbitol, 50 mM CaCl₂·2H₂O, and 8 g of phyto agar) in a diameter of about 5 mm and hardened.

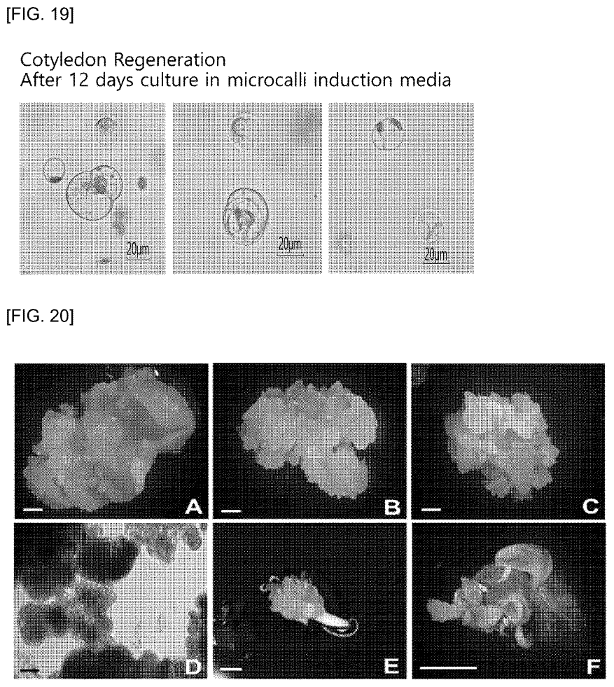

The hardened agar was collected and transferred to another petri dish, and then Medium E was added thereto to submerge the agar, followed by incubation under dark conditions. After formation of microcalli, the product was transferred to a chamber for culture under light conditions and the medium was replaced with Medium F. After about 4 to 6 weeks, upon formation of minicalli, the medium was replaced with Medium G to induce callus greening. When callus greening was induced, the calli was transferred to Medium H to induce shoots. Upon induction of shoots, the shoots were transferred to Medium I to induce roots. Upon induction of roots, the roots were transferred to Medium A to grow transfectants. The induction of minicalli is shown in FIG. 19 and the compositions of the media are as follows.

### <Medium F>

Per 1 liter, 2.70 g of MS modif. No. 4 (Duchefa), 107 mg of NH₄Cl, 1 ml of Vit. NN stock, 40 mg of adenine sulphate, 100 mg of casein hydrolysate, 2.5 g of sucrose, 54.7 g of mannitol, 0.1 mg of NAA, and 0.5 mg of BAP, pH 5.8 (KOH)

### <Medium G>

Per 1 liter, 2.70 g of MS modif. No. 4 (Duchefa), 267.5 mg of NH₄Cl, 1 ml of Vit. NN stock, 80 mg of adenine sulphate, 100 mg of casein hydrolysate, 2.5 g of sucrose, 36.4 g of mannitol, 0.1 mg of IAA, and 2.5 mg of Zeatine, pH 5.8 (KOH)

### <Medium H>

Per 1 liter, 4.4 g of MS salts and organics (Duchefa), 10 g of sucrose, 2 mg of Zeatin, 0.01 mg of NAA, 0.1 mg of GA3, and 2.5 g of Gelrite (Duchefa), pH 5.8 (KOH)

### <Medium I>

Per 1 liter, 4.4 g of MS salts and organics (Duchefa), 20 g of sucrose, 0.1 mg of GA3, and 2.5 g of Gelrite (Duchefa), pH 5.8 (KOH)

<Vitamins NN stock>

Per 50 ml, 100 mg of glycine, 5,000 mg of myo-inositol, 25 mg of thiamine-HCl, 25 mg of pyridoxine-HCl, 250 mg of nicotinic acid, 25 mg of folic acid, and 2.5 mg of biotin

### Example 8: Redifferentiation of Cannabis

FIG. 20 shows a process of embryogenic callus formation and Cannabis redifferentiation (A: white callus formed after 4 weeks from incubation in a 1 mgl⁻¹ 2,4-D MS medium, B: proliferation of the white callus, C: development of a globular embryogenic structure, D: embryogenic cell suspension cultures; E: development of somatic embryos from protoplast; and F: redifferentiated Cannabis stem. Scale bars 1 mm (A, B, C, E), 100 μm (D), and 1 cm (F))

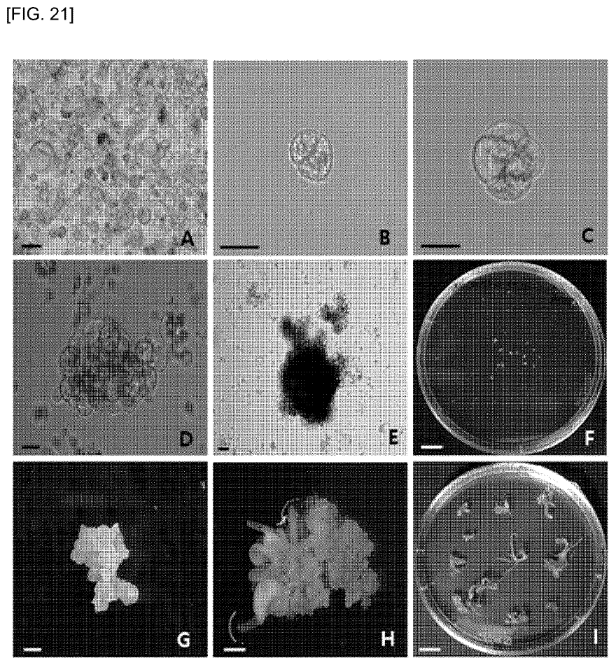

FIG. 21 shows a process of redifferentiation of Cannabis from Cannabis cell culture (A: isolated protoplasts; B: first division of protoplast cultured in a 1 mgl⁻¹ 2,4-D MS medium; C: second division of the protoplast cultured in the 1 mgl⁻¹ 2,4-D MS medium after one week; D: colony formed in the protoplast; E: micro callus; F: callus grown in a 1 mgl⁻¹ 2,4-D MS solid medium; G: globular somatic embryo grown from the callus; H: shoot grown from somatic embryo; and I: Cannabis grown from the protoplasts. Scale bars 50 μm (A-E), 1 mm (G-H), and 1 cm (F, I))

Based on the results described above, it was confirmed that redifferentiation was induced based on the results described above. The present invention discovered, for the first time, that an adult Cannabis plant may be prepared by inducing redifferentiation in Cannabis.

From the results described above, it was confirmed that the Cannabis gene may be edited by using Cas and gRNA, and redifferentiation up to Cannabis may be obtained via a process of inducing the callus.

The above description of the present invention is provided for the purpose of illustration, and it would be understood by those skilled in the art that various changes and modifications may be made without changing technical conception and essential features of the present invention. Thus, it is clear that the above-described embodiments are illustrative in all aspects and do not limit the present invention. Therefore, the scope of the disclosure is defined not by the detailed description, but by the claims and their equivalents, and all variations within the scope of the claims and their equivalents are to be construed as being included in the disclosure.

The invention claimed is:

1. A composition for editing a Cannabis gene comprising a gene editing protein and a guide RNA (gRNA).

2. The composition of claim 1, wherein the composition comprises the gene editing protein and the gRNA in the form of a protein: RNA ribonucleoprotein (RNP) complex.

3. The composition of claim 1, wherein the gene editing protein is TnpB, Cas12f, Cpf1, Cas9, or a functional analogue or variant thereof.

4. The composition of claim 1, wherein the gRNA comprises one sequence selected from the group consisting of SEQ ID NOS: 1 to 14 and 38 to 42 as a target sequence.

5. The composition of claim 1, wherein the gRNA comprises at least one sequence of SEQ ID NOS: 51 to 55 and 57 to 82.

6. The composition of claim 1, wherein the Cannabis gene comprises at least one of a gene encoding tetrahydrocannabinolic acid synthase (THCAS), a gene encoding phytoene desaturase (PDS), a gene encoding cannabidiolic acid synthase (CBDAS), and a gene encoding cannabichromenic acid synthase (CBCAS).

7. (canceled)

8. (canceled)

9. A method of editing Cannabis DNA, the method comprising culturing Cannabis protoplasts comprising a gene editing protein and a gRNA.

10. The method of claim 9, wherein the protoplasts are derived from Cannabis leaf mesophyll.

11. A method of preparing a gene-edited Cannabis, the method comprising:
culturing protoplasts isolated from Cannabis;
forming calli in the protoplasts;
forming somatic embryos in the calli; and
differentiating the somatic embryos into Cannabis mature plants.

12. The method of claim 11, wherein the protoplasts comprise a gene editing protein and a gRNA.

13. The method of claim 11, wherein genes of the protoplasts are edited by using a gene editing protein and a gRNA.

14. The method of claim 11, wherein components contained in the Cannabis are adjusted.

15. The method of claim 9, wherein the gene editing protein is TnpB, Cas12f, Cpf1, Cas9, or a functional analogue or variant thereof.

16. The method of claim 9, wherein the gRNA comprises one sequence selected from the group consisting of SEQ ID NOS: 1 to 14 and 38 to 42 as a target sequence, or
wherein the gRNA comprises at least one sequence of SEQ ID NOS: 51 to 55 and 57 to 82.

17. The method of claim 9, wherein the Cannabis gene comprises at least one of a gene encoding tetrahydrocannabinolic acid synthase (THCAS), a gene encoding phytoene desaturase (PDS), a gene encoding cannabidiolic acid synthase (CBDAS), and a gene encoding cannabichromenic acid synthase (CBCAS).

18. The method of claim 12, wherein the gene editing protein is TnpB, Cas12f, Cpf1, Cas9, or a functional analogue or variant thereof.

19. The method of claim 12, wherein the gRNA comprises one sequence selected from the group consisting of SEQ ID NOS: 1 to 14 and 38 to 42 as a target sequence, or
wherein the gRNA comprises at least one sequence of SEQ ID NOS: 51 to 55 and 57 to 82.

20. The method of claim 12, wherein the Cannabis gene comprises at least one of a gene encoding tetrahydrocannabinolic acid synthase (THCAS), a gene encoding phytoene desaturase (PDS), a gene encoding cannabidiolic acid synthase (CBDAS), and a gene encoding cannabichromenic acid synthase (CBCAS).


SEQUENCE LISTING (84 sequences)

SEQ ID NO: 1 - DNA, 24 bp, synthetic construct
  source 1..24: mol_type = other DNA
tttgtattgtcgaattcaggatag

SEQ ID NO: 2 - DNA, 24 bp, synthetic construct
  source 1..24: mol_type = other DNA
tttgttgtagtagacttgagaaac

SEQ ID NO: 3 - DNA, 25 bp, synthetic construct
  source 1..25: mol_type = other DNA
tttgatcgaatgcatgtttctcaag

SEQ ID NO: 4 - DNA, 23 bp, synthetic construct
  source 1..23: mol_type = other DNA
tttattattggatcaatgagaag

SEQ ID NO: 5 - DNA, 23 bp, synthetic construct
  source 1..23: mol_type = other DNA
tttagtggaggaggctatggagc

SEQ ID NO: 6 - DNA, 24 bp, synthetic construct
  source 1..24: mol_type = other DNA
tttatgcctcctggtacaagactg

SEQ ID NO: 7 - DNA, 24 bp, synthetic construct
  source 1..24: mol_type = other DNA
tttgtgtggattatccaagaccag

SEQ ID NO: 8 - DNA, 24 bp, synthetic construct
  source 1..24: mol_type = other DNA
tttatctactgcaaaatacttggc

SEQ ID NO: 9 - DNA, 25 bp, synthetic construct
  source 1..25: mol_type = other DNA
tttgcaatgcccagcaagccaggag

SEQ ID NO: 10 - DNA, 25 bp, synthetic construct
  source 1..25: mol_type = other DNA
tttgatttcaccgatgctctgccag

SEQ ID NO: 11 - DNA, 24 bp, synthetic construct
  source 1..24: mol_type = other DNA
tttacggaacaatgagatgctgac

SEQ ID NO: 12 - DNA, 27 bp, synthetic construct
  source 1..27: mol_type = other DNA
tttgcaattgggcttctgcctgcaatg

SEQ ID NO: 13 - DNA, 24 bp, synthetic construct
  source 1..24: mol_type = other DNA
tttgacggtgaaaccatcttgagc

SEQ ID NO: 14 - DNA, 23 bp, synthetic construct
  source 1..23: mol_type = other DNA
ttcttcagtcttgtaccaggagg

SEQ ID NO: 15 - DNA, 102 bp, synthetic construct
  source 1..102: mol_type = other DNA
atacgactcactatagaccgcttcacttagagtgaaggtgggctgcttgcatcagcctaatgtcgagaagtgctttcttcggaaagtaaccctcgaaacaaa

SEQ ID NO: 16 - DNA, 37 bp, synthetic construct
  source 1..37: mol_type = other DNA
gttgcattcctttctttgtttcgagggttactttccg

SEQ ID NO: 17 - DNA, 27 bp, synthetic construct
  source 1..27: mol_type = other DNA
gaaattaatacgactcactatagaccg

SEQ ID NO: 18 - DNA, 11 bp, synthetic construct
  source 1..11: mol_type = other DNA
aaaaaataaaa

SEQ ID NO: 19 - DNA, 21 bp, synthetic construct
  source 1..21: mol_type = other DNA
gttgcattcctttctttgttt

SEQ ID NO: 20 - DNA, 77 bp, synthetic construct
  source 1..77: mol_type = other DNA
agcaccgactcggtgccactttttcaagttgataacggactagccttattttaacttgctatttctagctctaaaac

SEQ ID NO: 21 - DNA, 18 bp, synthetic construct
  source 1..18: mol_type = other DNA
aatacgactcactatagg

SEQ ID NO: 22 - DNA, 23 bp, synthetic construct
  source 1..23: mol_type = other DNA
gttttagagctagaaatagcaag

SEQ ID NO: 23 - DNA, 23 bp, synthetic construct
  source 1..23: mol_type = other DNA
aatttaatacgactcactatagg

SEQ ID NO: 24 - DNA, 17 bp, synthetic construct
  source 1..17: mol_type = other DNA
aaaagcaccgactcggt

SEQ ID NO: 25 - DNA, 1638 bp, synthetic construct
  source 1..1638: mol_type = other DNA
atgaattgctcagcattttccttttggtttgtttgcaaaataatatttttctttctctcattccatatccaaatttcaatagctaatcctcgagaaaacttccttaaatgcttctcaaaacatattcccaacaatgtagcaaatccaaaactcgtatacactcaacacgaccaattgtatatgtctatcctgaattcgacaatacaaaatcttagattcatctctgatacaaccccaaaaccactcgttattgtcactccttcaaataactcccatatccaagcaactattttatgctctaagaaagttggcttgcagattcgaactcgaagcggtggccatgatgctgagggtatgtcctacatatctcaagtcccatttgttgtagtagacttgagaaacatgcattcgatcaaaatagatgttcatagccaaactgcgtgggttgaagccggagctacccttggagaagtttattattggatcaatgagaagaatgagaatcttagttttcctggtgggtattgccctactgttggcgtaggtggacactttagtggaggaggctatggagcattgatgcgaaattatggccttgcggctgataatattattgatgcacacttagtcaatgttgatggaaaagttctagatcgaaaatccatgggagaagatctgttttgggctatacgtggtggtggaggagaaaactttggaatcattgcagcatggaaaatcaaactggttgctgtcccatcaaagtctactatattcagtgttaaaaagaacatggagatacatgggcttgtcaagttatttaacaaatggcaaaatattgcttacaagtatgacaaagatttagtactcatgactcacttcataacaaagaatattacagataatcatgggaagaataagactacagtacatggttacttctcttcaatttttcatggtggagtggatagtctagtcgacttgatgaacaagagctttcctgagttgggtattaaaaaaactgattgcaaagaatttagctggattgatacaaccatcttctacagtggtgttgtaaattttaacactgctaattttaaaaaggaaattttgcttgatagatcagctgggaagaagacggctttctcaattaagttagactatgttaagaaaccaattccagaaactgcaatggtcaaaattttggaaaaattatatgaagaagatgtaggagctgggatgtatgtgttgtacccttacggtggtataatggaggagatttcagaatcagcaattccattccctcatcgagctggaataatgtatgaactttggtacactgcttcctgggagaagcaagaagataatgaaaagcatataaactgggttcgaagtgtttataattttacgactccttatgtgtcccaaaatccaagattggcgtatctcaattatagggaccttgatttaggaaaaactaatcatgcgagtcctaataattacacacaagcacgtatttggggtgaaaagtattttggtaaaaattttaacaggttagttaaggtgaaaactaaagttgatcccaataatttttttagaaacgaacaaagtatcccacctcttccaccgcatcatcattaa

SEQ ID NO: 26 - DNA, 5370 bp, synthetic construct
  source 1..5370: mol_type = other DNA
atgtctcagtggggttctgtttcagctactaacttcagctcccaccagagtcgtatctttgaatttccaaacgtaggaacagtacccagatgttgtttttcattgggttccaaaaaaatggcgtctttggcttttgggtctaatcaatttatgcctcctggtacaagactgaagaagaagaagggtgttttgcctttaaaggtatttttgtgttgaatttgaatgtttttcttgttgggatgaaagaaaaatgagaaatttattaactgaattgagttctgaaataggtggtttgtgtggattatccaagaccagagcttgatagtactgttaactttttggaagctgcggctttgtctgcttcttttcgtggctctcctcgtccagctaagcctttgaaagttgtaattgctggtgcaggtgaagaaagattgcatttttatttacttaatgattttttgtgtttcttacttttgagtattgtttgcttgagaataggggagaataatcaagtttaagagaaatctttacttataataagaaagttcaattagcctattttttataattaagaaagatttgatatgaaaatatgtattctgtttttttatcttaattatactgaattttgtaactaatcttgtctattcgagtcactttggtgtgaagcttgcgattcgagttgtattatcgtgtttgactcatcaacatcatcaagtttacctgcttgtaataaaaaacattacttataataagaaagttcaattagcttattttttttttttcattttatttgtatgcaaatgattaaaaaagatttggtgtgaaaatgtttattttgcttttatcttaattttactcaattttgtaactaatcttgtctgttagagtcacctcagtgtgaagagtgtgaatcgttttcgagttgtattatcattttgtaaagcttttatgattttgtatatgtatggtgagtccttcgctaattgttttctcaaattcttgtaggtttggctggtttatctactgcaaaatacttggcagatgcaggtcataaaccgatattactcgaagcaagagatgttttaggtggaaaggtttttacactgatctagtttaccccccatgttaacatcaaacttattctttcattacaattcattttcattttcacttttcaattcaaggacaaactcatttttgcttatcttatagtttacatttcaatcctttttgttgggtagtttcgaaatagttaaactatgagagtgttttttctgattagaaatctttgccctttaggtggcagcatggaaagatgatgacggtgactggtatgagaccggtctacatatattctgtaagttgaatctcggaaccatttcattttaccaaattcagttttaccccctttgaattttcgagtgcttattttctttgtatgcgtcgcaagttggagcttatcccaacgtacagaacttgttcggagagctaggtattgatgatcggttgcaatggaaagaacattcaatgatctttgcaatgcccagcaagccaggagaattcagccgatttgatttcaccgatgctctgccagctcccttaaatggtaagagactacatcatgaccaacctatctctgtactttataactaagaaaatggggttttcctccatggtggagagtaactatgtttttgaaactagttggtcaattactcaattatgtgcatttgaaatcagaatgtgttttgtcgaattctgcaatgcgtggatacgctattgttcaaactgggtgttcttgaaaccttgtaaattctacctctttaaatccaagtattcccacaaaagctgttttgttggcatttttggtgtttaacagtttttgtgctcatttggcaggactgatgttcttgtaaccttattctttgcttctgttaagttttttgtttcctttcggaaattttactatacactatcttgtttttcctaggaatatgggccattttacggaacaatgagatgctgacctgggcagaaaaagtcaaatttgcaattgggcttctgcctgcaatggtaggcggacagccttatgtcgaagctcaagatggtttcaccgtcaaagattggatgagaaagcaggtaaaataatggaagaagaaattcttttataattgtaagtttgtaaaagtattcaaagggattcattttatggtctcatgtgaatttttgaagtttcaagttcgaagagtgatttatgatgtttctgttttctttcagggcatacctgatcgtgtaacggatgaggtcttcattgccatgtccaaggccttaaacttcattaacccagatgaactttcaatgcaatgtatattgattgctttaaaccgatttcttcaggtatgttgtttacatttatccttctaggttgatatatcatatcgataccataattttattttattcccgctcatataaacaaagaaaggaatctagaggttaagttactatgcctaaagttactatttcctatcagattgtggaatttggggacggttaggattccgcattgaccaattatgagattagttagtagcatataactatttgggcaggctagcgtttgggagtgagttagtggttgtattaagtagtatcaaagccatgcaccctcaagtgggaccaacgtcgcgggatgggcgacttgtggatcaagcctagtgtgggacaccgcagagtaggctaccgtagagtgggctcccatgggcgtaagctccaaagagattggttagtagtatatagctatttggacaccctcgtctcttaggttaatttttgggaatgagttctacccgagtggtttcataagactgctatgatctgtttaccataaagtatttgaagcaatagtgttaaaagtagtcaatgtaccaaactaaaaatttcgaagatttgaatggagggaggttaagtaatacttatacactttttgtacgggtacaatttttaatgccttgaagggatcatttcttcttaggttatgaacattttgatactactgtttttgtcataaaaggaagaaacttatttaagccaaatactttttgatgagttttttgtggaatgttacattgaaattaaatgaacaggagaagcatggttccaagatggctttcctagatggaaatcccccagagagactctgtatgcccatagtcgatcatatccagtcattgggcggtgaagtccgacttaattcccgtatacaaaaaatcgacctgaatgatgatggaactgtaaagagatttttactaactaatggtagtgaaatcgaaggggatgcatatgtgtttgcaacaccaggtatgttttcgacccttgttttcccgtccgatataaagtttgtattgagttcaaaatcataatatctctaatgatatcaacaacttacattccaaacagttgatatcctcaagcttctattgcccgaaaactggaaagagattccgtatttcaagaaattggataaattagttggcgtccccgttattaatgtccacatatggttagtaacagactctctctctctctgtctctccttaatcaagcatagaaaaattcagaagttagatagaggtaggtggtctttgattcatggattgaccaaatttatgaatatttttcttcaagtaggtatcttaataatttctaaagattcaattttatcttaaccatttgtttgaagcaattttatataatattttcttgataaatttacatattaagttatgtttatcatgtgtatgtgtgaataatatgatcattaatattttgtaaacaagttttctattgtttggtggtaaaaatgttttctttgttgcaggttcgaccgaaaattgaagaacacatatgatcaccttcttttcagcaggttctgatactttacttcttaattttacccaatttcattttaaaaatggaacctttatgctagctttaaatgcatattttatcgaattaatagttacaaacatcttctactcaagtagtttcccgatcaggcgtgatttaccgatcattccatagtacctagtactacatagcaattgttatagtttcccgatctaaaagtattgtagggttaggcacagatcctagttacaaacatgttccgtgtttagtgcttctaatgctgcaaatgagatcacatattaatttgccaaaagaaaatgaaaaagggaaagatcgtttcttattaatgcttgtaccgacttcaaattaacgtcaatatgcttctttttgctgtgtttgattcggtaaattaacttatttcttccccatgcagaagtcctctcttgagtgtctacgccgatatgtcggtaacttgtaaggtaataactactttcttaaaatgtcatttgaggctaaaattataacactttcattcataataataaaagttagtaatcacaatttttactttttaatatttagtttatattccacttgtatattctaggaatattacaatccaaaccagtctatgctagagttggttttcgcaccagcagaagaatggatttcgcgtagtgactcagatattatcgatgctacgatgaaggagctttctaagctctttcctgatgaaatagcagctgatcagagcaaagcgaaaatcttaaagtaccatgttgtaaaaacaccgaggttcgatttttttcaccactaatttgaactattatgagctcgtttggtacaagatttacttctactaatagaagttttagttattgtttttcttctttagaagagcttcgcaagtaattatttacttttatttgagtagaataaggataaaaatcgaaaattttgttagagactagtgaagatttacctctctaagaagagtttccaaacacgaaaacaagatcattttttcttttaagaaatttttagttggaaaactttgatgcaaaggaaatatgtttttagttttcgggtatggctttgctgtgagtagttcagttactaaatttctcttctgaatctgattcatatcaggtctgtctacaaaactgttcctgattgcgaaccttgtcgccccagacaaagatctcctcttgaaggtttctatttagcaggtgactacacaaaacaaaaatatttggcttcaatggaaggtgcggttttgtcaggaaagttttgtgcacaagcaattgtacaggtaatttaactcggcaagttaagaaggcgtttttgtttgctgctctcaatgaaataactccctttgtaatgatactttactttcaggattatgagttgcttgctgctcggggtcagagaattttggccgaggctggaagtcgttga

SEQ ID NO: 27 - protein, 557 aa, synthetic construct
MGEKSSRRRRNGKSGAWTAAITSCVGGKMAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSDVCYTRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKIGEKSAWMLNLSIDVPKIDKGVDPSIIGGIDVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENADYNAALNISNPKLKSTKEEP

SEQ ID NO: 28 - protein, 555 aa, synthetic construct
MEKRINKIRKKLSADNATKPVSRSGPMAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSDVCYTRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKIGEKSAWMLNLSIDVPKIDKGVDPSIIGGIDVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENADYNAALNISNPKLKSTKEEP

SEQ ID NO: 29 - protein, 557 aa, synthetic construct
MAGGPGAGSAAPVSSTSSLPLAALNMRVMAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSDVCYTRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKIGEKSAWMLNLSIDVPKIDKGVDPSIIGGIDVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENADYNAALNISNPKLKSTKEEP

SEQ ID NO: 30 - protein, 555 aa, synthetic construct
MAGGPGAGSAAPVSSTSSLPLAALNMMAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSDVCYTRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKIGEKSAWMLNLSIDVPKIDKGVDPSIIGGIDVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENADYNAALNISNPKLKSTKEEP

SEQ ID NO: 31 - protein, 529 aa, synthetic construct
MAKNTITKTLKLRIVRPYNSAEVEKIVADEKNNREKIALEKNKDKVKEACSKHLKVAAYCTTQVERNACLFCKARKLDDKFYQKLRGQFPDAVFWQEISEIFRQLQKQAAEIYNQSLIELYYEIFIKGKGIANASSVEHYLSDVCYTRAAELFKNAAIASGLRSKIKSNFRLKELKNMKSGLPTTKSDNFPIPLVKQKGGQYTGFEISNHNSDFIIKIPFGRWQVKKEIDKYRPWEKFDFEQVQKSPKPISLLLSTQRRKRNKGWSKDEGTEAEIKKVMNGDYQTSYIEVKRGSKIGEKSAWMLNLSIDVPKIDKGVDPSIIGGIDVGVKSPLVCAINNAFSRYSISDNDLFHFNKKMFARRRILLKKNRHKRAGHGAKNKLKPITILTEKSERFRKKLIERWACEIADFFIKNKVGTVQMENLESMKRKEDSYFNIRLRGFWPYAEMQNKIEFKLKQYGIEIRKVAPNNTSKTCSKCGHLNNYFNFEYRKKNKFPHFKCEKCNFKENADYNAALNISNPKLKSTKEEP

SEQ ID NO: 32 - RNA, 222 nt, synthetic construct
  source 1..222: mol_type = other RNA; note = n=a,u,g,or c
cttcactgataaagtggagaaccgcttcaccaaaagctgtcccttaggggattagaacttgagtgaaggtgggctgcttgcatcagcctaatgtcgagaagtgctttcttcggaaagtaaccctcgaaacaaattcatttttcctctccaattctgcacaagaaagttgcagaacccgaatagacgaatgaaggaatgcaacnnnnnnnnnnnnnnnnnnnn

SEQ ID NO: 33 - RNA, 30 nt, synthetic construct
  source 1..30: mol_type = other RNA; note = n=a,u,g,or c
gttgcagaacccgaatagnnnnntgaagga

SEQ ID NO: 34 - RNA, 182 nt, unidentified
  source 1..182: mol_type = other RNA
accgcttcaccaaaagctgtcccttaggggattagaacttgagtgaaggtgggctgcttgcatcagcctaatgtcgagaagtgctttcttcggaaagtaaccctcgaaacaaattcagtgctcctctccaattctgcacaagaaagttgcagaacccgaatagagcaatgaaggaatgcaac

SEQ ID NO: 35 - RNA, 127 nt, synthetic construct
  source 1..127: mol_type = other RNA
accgcttcaccaaaagctgtcccttaggggattagaacttgagtgaaggtgggctgcttgcatcagcctaatgtcgagaagtgctttcttcggaaagtaaccctcgaaacaaagaaaggaatgcaac

SEQ ID NO: 36 - DNA, 100 bp, synthetic construct
  source 1..100: mol_type = other DNA
accgcttcacttagagtgaaggtgggctgcttgcatcagcctaatgtcgagaagtgctttcttcggaaagtaaccctcgaaacaaagaaaggaatgcaac

SEQ ID NO: 37 - DNA, 1635 bp, synthetic construct
  source 1..1635: mol_type = other DNA
atgaagtgctcaacattctccttttggtttgtttgcaagataatatttttctttttctcattcaatatccaaacttccattgctaatcctcgagaaaacttccttaaatgcttctcgcaatatattcccaataatgcaacaaatctaaaactcgtatacactcaaaacaacccattgtatatgtctgtcctaaattcgacaatacacaatcttagattcacctctgacacaaccccaaaaccacttgttatcgtcactccttcacatgtctctcatatccaaggcactattctatgctccaagaaagttggcttgcagattcgaactcgaagtggtggtcatgattctgagggcatgtcctacatatctcaagtcccatttgttatagtagacttgagaaacatgcgttcaatcaaaatagatgttcatagccaaactgcatgggttgaagccggagctacccttggagaagtttattattgggttaatgagaaaaatgagaatcttagtttggcggctgggtattgccctactgtttgcgcaggtggacactttggtggaggaggctatggaccattgatgagaaactatggcctcgcggctgataatatcattgatgcacacttagtcaacgttcatggaaaagtgctagatcgaaaatctatgggggaagatctcttttgggctttacgtggtggtggagcagaaagcttcggaatcattgtagcatggaaaattagactggttgctgtcccaaagtctactatgtttagtgttaaaaagatcatggagatacatgagcttgtcaagttagttaacaaatggcaaaatattgcttacaagtatgacaaagatttattactcatgactcacttcataactaggaacattacagataatcaagggaagaataagacagcaatacacacttacttctcttcagttttccttggtggagtggatagtctagtcgacttgatgaacaagagttttcctgagttgggtattaaaaaaacggattgcagacaattgagctggattgatactatcatcttctatagtggtgttgtaaattacgacactgataattttaacaaggaaattttgcttgatagatccgctgggcagaacggtgctttcaagattaagttagactacgttaagaaaccaattccagaatctgtatttgtccaaattttggaaaaattatatgaagaagatataggagctgggatgtatgcgttgtacccttacggtggtataatggatgagatttcagaatcagcaattccattccctcatcgagctggaatcttgtatgagttatggtacatatgtagttgggagaagcaagaagataacgaaaagcatctaaactggattagaaatatttataacttcatgactccttatgtgtccaaaaatccaagattggcatatctcaattatagagaccttgatataggaataaatgatcccaagaatccaaataattacacacaagcacgtatttggggtgagaagtattttggtaaaaattttgacaggctagtaaaagtgaaaaccctggttgatcccaataacttttttagaaacgaacaaagcatcccacctcttccacggcatcgtcattaa

SEQ ID NO: 38 - DNA, 26 bp, synthetic construct
  source 1..26: mol_type = other DNA
tttgttgcattattgggaatatattg

SEQ ID NO: 39 - DNA, 24 bp, synthetic construct
  source 1..24: mol_type = other DNA
tttagatttgttgcattattggga

SEQ ID NO: 40 - DNA, 23 bp, synthetic construct
  source 1..23: mol_type = other DNA
tttgagtgtatacgagttttaga

SEQ ID NO: 41 - DNA, 25 bp, synthetic construct
  source 1..25: mol_type = other DNA
tttggggttgtgtcagaggtgaatc

SEQ ID NO: 42 - DNA, 24 bp, synthetic construct
  source 1..24: mol_type = other DNA
tttgattgaacgcatgtttctcaa

SEQ ID NO: 43 - DNA, 1638 bp, synthetic construct
  source 1..1638: mol_type = other DNA
atgaattgctcaacattctccttttggtttgtttgcaaaataatatttttctttctctcattcaatatccaaatttcaatagctaatcctcaagaaaacttccttaaatgcttctcggaatatattcctaacaatccagcaaatccaaaattcatatacactcaacacgaccaattgtatatgtctgtcctgaattcgacaatacaaaatcttagattcacctctgatacaaccccaaaaccactcgttattgtcactccttcaaatgtctcccatatccaggtcagtattctctgctccaagaaagttggtttgcagattcgaactcgaagcggtggccatgatgctgagggtttgtcctacatatctcaagtcccatttgctatagtagacttgagaaacatgcatacggtcaaagtagatattcatagccaaactgcgtgggttgaagccggagctacccttggagaagtttattattggatcaatgagatgaatgagaattttagttttcctggtgggtattgccctactgttggcgtaggtggacactttagtggaggaggctatggagcattgatgcgaaattatggccttgcggctgataatatcattgatgcacacttagtcaatgttgatggaaaagttctagatcgaaaatccatgggagaagatctattttgggctatacgtggtggaggaggagaaaactttggaatcattgcagcatggaaaatcaaacttgttgttgtcccatcaaaggctactatattcagtgttaaaaagaacatggagatacatgggcttgtcaagttatttaacaaatggcaaaatattgcttacaagtatgacaaagatttaatgctcacgactcacttcagaactaggaatattacagataatcatgggaagaataagactacagtacatggttacttctcttccatttttcttggtggagtggatagtctagttcacttgatgaacaagagctttcctgagttgggtattaaaaaaactgattgcaaagaattgagctggattgatacaaccatcttctacagtggtgttgtaaattacaacactgctaattttaaaaaggaaattttgcttgatagatcagctgggaagaagacggctttctcaattaagttagactatgttaagaaactaatacctgaaactgcaatggtcaaaattttggaaaaattatatgaagaagaggtaggagttgggatgtatgtgttgtacccttacggtggtataatggatgagatttcagaatcagcaattccattccctcatcgagctggaataatgtatgaactttggtacactgctacctgggagaagcaagaagataacgaaaagcatataaactgggttcgaagtgtttataatttcacaactccttatgtgtcccaaaatccaagattggcgtatctcaattatagggaccttgatttaggaaaaactaatcctgagagtcctaataattacacacaagcacgtatttggggtgaaaagtattttggtaaaaattttaacaggttagttaaggtgaaaaccaaagctgatcccaataatttttttagaaacgaacaaagtatcccacctcttccaccgcgtcatcattaa

SEQ ID NO: 44 - protein, 1368 aa, synthetic construct [sequence omitted]

SEQ ID NO: 45 - protein, 1307 aa, synthetic construct
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRN

SEQ ID NO: 46 - DNA, 20 bp, synthetic construct
  source 1..20: mol_type = other DNA
atctacaagagtagaaatta

SEQ ID NO: 47 - DNA, 43 bp, synthetic construct
  source 1..43: mol_type = other DNA
gaaattaatacgactcactatagtaatttctactcttgtagat

SEQ ID NO: 48 - DNA, 23 bp, synthetic construct
  source 1..23: mol_type = other DNA
gaaattaatacgactcactatag

SEQ ID NO: 49 - DNA, 11 bp, synthetic construct
  source 1..11: mol_type = other DNA
aaaaaataaaa

SEQ ID NO: 50 - RNA, 20 nt, synthetic construct
  source 1..20: mol_type = other RNA
taatttctactcttgtagat

SEQ ID NO: 51 - RNA, 51 nt, synthetic construct
  source 1..51: mol_type = other RNA
taatttctactcttgtagattattgtcgaattcaggatagttttatttttt

SEQ ID NO: 52 - RNA, 51 nt, synthetic construct
  source 1..51: mol_type = other RNA
taatttctactcttgtagatttgtagtagacttgagaaacttttatttttt

SEQ ID NO: 53 - RNA, 52 nt, synthetic construct
  source 1..52: mol_type = other RNA
taatttctactcttgtagatatcgaatgcatgtttctcaagttttatttttt

SEQ ID NO: 54 - RNA, 50 nt, synthetic construct
  source 1..50: mol_type = other RNA
taatttctactcttgtagatttattggatcaatgagaagttttatttttt

SEQ ID NO: 55 - RNA, 50 nt, synthetic construct
  source 1..50: mol_type = other RNA
taatttctactcttgtagatgtggaggaggctatggagcttttatttttt

SEQ ID NO: 56 - RNA, 100 nt, synthetic construct
  source 1..100: mol_type = other RNA
accgcttcacttagagtgaaggtgggctgcttgcatcagcctaatgtcgagaagtgctttcttcggaaagtaaccctcgaaacaaagaaaggaatgcaac

SEQ ID NO: 57 - RNA, 131 nt, synthetic construct
  source 1..131: mol_type = other RNA
accgcttcacttagagtgaaggtgggctgcttgcatcagcctaatgtcgagaagtgctttcttcggaaagtaaccctcgaaacaaagaaaggaatgcaactattgtcgaattcaggatagttttatttttt

SEQ ID NO: 58 - RNA, 131 nt, synthetic construct
  source 1..131: mol_type = other RNA
accgcttcacttagagtgaaggtgggctgcttgcatcagcctaatgtcgagaagtgctttcttcggaaagtaaccctcgaaacaaagaaaggaatgcaacttgtagtagacttgagaaacttttatttttt

SEQ ID NO: 59 - RNA, 132 nt, synthetic construct
  source 1..132: mol_type = other RNA
accgcttcacttagagtgaaggtgggctgcttgcatcagcctaatgtcgagaagtgctttcttcggaaagtaaccctcgaaacaaagaaaggaatgcaacatcgaatgcatgtttctcaagttttatttttt

SEQ ID NO: 60 - RNA, 130 nt, synthetic construct
  source 1..130: mol_type = other RNA
accgcttcacttagagtgaaggtgggctgcttgcatcagcctaatgtcgagaagtgctttcttcggaaagtaaccctcgaaacaaagaaaggaatgcaacttattggatcaatgagaagttttatttttt

SEQ ID NO: 61 - RNA, 130 nt, synthetic construct
  source 1..130: mol_type = other RNA
accgcttcacttagagtgaaggtgggctgcttgcatcagcctaatgtcgagaagtgctttcttcggaaagtaaccctcgaaacaaagaaaggaatgcaacgtggaggaggctatggagcttttatttttt

SEQ ID NO: 62 - RNA, 51 nt, synthetic construct
  source 1..51: mol_type = other RNA
taatttctactcttgtagattgcctcctggtacaagactgttttatttttt

SEQ ID NO: 63 - RNA, 51 nt, synthetic construct
  source 1..51: mol_type = other RNA
taatttctactcttgtagattgtggattatccaagaccagttttatttttt

SEQ ID NO: 64 - RNA, 51 nt, synthetic construct
  source 1..51: mol_type = other RNA
taatttctactcttgtagattctactgcaaaatacttggcttttatttttt

SEQ ID NO: 65 - RNA, 52 nt, synthetic construct
  source 1..52: mol_type = other RNA
taatttctactcttgtagatcaatgcccagcaagccaggagttttatttttt

SEQ ID NO: 66 - RNA, 52 nt, synthetic construct
  source 1..52: mol_type = other RNA
taatttctactcttgtagatatttcaccgatgctctgccagttttatttttt

SEQ ID NO: 67 - RNA, 51 nt, synthetic construct
  source 1..51: mol_type = other RNA
taatttctactcttgtagatcggaacaatgagatgctgacttttatttttt

SEQ ID NO: 68 - RNA, 54 nt, synthetic construct
  source 1..54: mol_type = other RNA
taatttctactcttgtagatcaattgggcttctgcctgcaatgttttatttttt

SEQ ID NO: 69 - RNA, 51 nt, synthetic construct
  source 1..51: mol_type = other RNA
taatttctactcttgtagatacggtgaaaccatcttgagcttttatttttt

SEQ ID NO: 70 - RNA, 131 nt, synthetic construct
  source 1..131: mol_type = other RNA
accgcttcacttagagtgaaggtgggctgcttgcatcagcctaatgtcgagaagtgctttcttcggaaagtaaccctcgaaacaaagaaaggaatgcaactgcctcctggtacaagactgttttatttttt

SEQ ID NO: 71 - RNA, 131 nt, synthetic construct
  source 1..131: mol_type = other RNA
accgcttcacttagagtgaaggtgggctgcttgcatcagcctaatgtcgagaagtgctttcttcggaaagtaaccctcgaaacaaagaaaggaatgcaactgtggattatccaagaccagttttatttttt

SEQ ID NO: 72 - RNA, 131 nt, synthetic construct
  source 1..131: mol_type = other RNA
accgcttcacttagagtgaaggtgggctgcttgcatcagcctaatgtcgagaagtgctttcttcggaaagtaaccctcgaaacaaagaaaggaatgcaactctactgcaaaatacttggcttttatttttt

SEQ ID NO: 73 - RNA, 132 nt, synthetic construct
  source 1..132: mol_type = other RNA
accgcttcacttagagtgaaggtgggctgcttgcatcagcctaatgtcgagaagtgctttcttcggaaagtaaccctcgaaacaaagaaaggaatgcaaccaatgcccagcaagccaggagttttatttttt

SEQ ID NO: 74 - RNA, 132 nt, synthetic construct
  source 1..132: mol_type = other RNA
accgcttcacttagagtgaaggtgggctgcttgcatcagcctaatgtcgagaagtgctttcttcggaaagtaaccctcgaaacaaagaaaggaatgcaacatttcaccgatgctctgccagttttatttttt

SEQ ID NO: 75 - RNA, 131 nt, synthetic construct
  source 1..131: mol_type = other RNA
accgcttcacttagagtgaaggtgggctgcttgcatcagcctaatgtcgagaagtgctttcttcggaaagtaaccctcgaaacaaagaaaggaatgcaaccggaacaatgagatgctgacttttatttttt

SEQ ID NO: 76 - RNA, 134 nt, synthetic construct
  source 1..134: mol_type = other RNA
accgcttcacttagagtgaaggtgggctgcttgcatcagcctaatgtcgagaagtgctttcttcggaaagtaaccctcgaaacaaagaaaggaatgcaaccaattgggcttctgcctgcaatgttttatttttt

SEQ ID NO: 77 - RNA, 131 nt, synthetic construct
  source 1..131: mol_type = other RNA
accgcttcacttagagtgaaggtgggctgcttgcatcagcctaatgtcgagaagtgctttcttcggaaagtaaccctcgaaacaaagaaaggaatgcaacacggtgaaaccatcttgagcttttatttttt

SEQ ID NO: 78 - RNA, 53 nt, synthetic construct
  source 1..53: mol_type = other RNA
taatttctactcttgtagatttgcattattgggaatatattgttttatttttt

SEQ ID NO: 79 - RNA, 51 nt, synthetic construct
  source 1..51: mol_type = other RNA
taatttctactcttgtagatgatttgttgcattattgggattttatttttt

SEQ ID NO: 80 - RNA, 50 nt, synthetic construct
  source 1..50: mol_type = other RNA
taatttctactcttgtagatagtgtatacgagttttagattttatttttt

SEQ ID NO: 81 - RNA, 52 nt, synthetic construct
  source 1..52: mol_type = other RNA
taatttctactcttgtagatgggttgtgtcagaggtgaatcttttatttttt

SEQ ID NO: 82 - RNA, 51 nt, synthetic construct
  source 1..51: mol_type = other RNA
taatttctactcttgtagatattgaacgcatgtttctcaattttatttttt

SEQ ID NO: 83 - RNA, 80 nt, synthetic construct
  source 1..80: mol_type = other RNA
gttttagagctagaaatagcaagttaaaataaggctagtccgttatcaacttgaaaaagtggcaccgagtcggtgctttt

SEQ ID NO: 84 - RNA, 100 nt, synthetic construct
  source 1..100: mol_type = other RNA
ttcttcagtcttgtaccagggttttagagctagaaatagcaagttaaaataaggctagtccgttatcaacttgaaaaagtggcaccgagtcggtgctttt